(19) United States
(12) Patent Application Publication
(10) Pub. No.: US 2004/0043950 A1
(43) Pub. Date: Mar. 4, 2004

(54) WT1 antisense oligos for the inhibition of breast cancer

(75) Inventors: Gabriel Lopez-Berestein, Bellaire, TX (US); Ana Maria Tari, Houston, TX (US); Pablo Zapata-Benavides, Guadalupe NL (MX)

Correspondence Address: Priya D. Subramony, Fulbright & Jaworski L.L.P., Suite 2400, 600 Congress Avenue, Austin, TX, 78701, US

(73) Assignee: Board of Regents, The University of Texas System

(21) Appl. No.: 10/336,253

(22) Filed: Jan. 3, 2003

Related U.S. Application Data
(60) Provisional application No. 60/345,102, filed on Jan. 3, 2002.

(51) Int. Cl. A61K 48/00

(52) U.S. Cl. 514/44

(57) ABSTRACT

The present invention provides methods for inhibiting the growth of breast cancer cells and methods for treating breast cancers expressing Wilms' Tumor 1 (WT1) gene product using a WT1 antisense oligonucleotide. It further provides methods of predicting breast cancer progression and methods for the screening of candidate substances for activity against breast cancer.

The present application claims priority to provisional U.S. Patent Application Serial No. 60/345,102 filed Jan. 3, 2002. The entire text of the above referenced applications are incorporated herein by reference and without disclaimer.

### BACKGROUND OF THE INVENTION

1. Field of the Invention

The present invention relates generally to the fields of cancer therapy, specifically treatment of breast cancer. More particularly, these treatments involve the use of antisense oligonucleotides against the Wilms' Tumor 1 (WT1) gene, and lipid associated and liposomal formulations thereof.

2. Description of Related Art

Breast cancer is the second most common form of cancer among women in the U.S., and the second leading cause of cancer deaths among women. Although several forms of radiation-therapy and chemotherapy are available for the treatment of such cancers, these therapies, especially when used in high doses, have side effects such as killing non-cancerous cells. When used in lower doses, they may not be enough to eradicate the cancer completely. Gene therapy is another form of anti-cancer therapy that has been receiving much attention. However, for a gene therapy to be effective it is necessary to identify genes and gene products that are involved in the disease and may be targeted for therapy.

Wilms' Tumor is a pediatric kidney cancer arising from pluripotent embryonic renal precursors (Lee et al., 2001). WT1 is a Wilms' Tumor gene that was isolated from chromosome 11p13 by a positional cloning technique (Call et al., 1990; Gessler et al., 1990). Abnormalities of the WT1 gene are found in approximately 10% of patients with Wilms' tumor and the WT1 has been categorized to be a tumor suppressor gene (Haber et al., 1990; Little et al., 1992).

It has been shown that WT1 participates in leukemogenesis and all leukemic cells express high levels of WT1 expression (Inoue et al., 1994). It has also been shown that a WT1 antisense oligomer suppresses and inhibits growth of leukemia cells (U.S. Pat. No. 6,034,235; Yamagami et al., 1996).

Oji et al., (1999), have determine the role of the Wilms' tumor gene WT1 in tumorigenesis of solid tumors, by examining the expression of the WT1 gene in 34 solid tumor cell lines including four gastric cancer cell lines, five colon cancer cell lines, 15 lung cancer cell lines, four breast cancer cell lines, one germ cell tumor cell line, two ovarian cancer cell lines, one uterine cancer cell line, one thyroid cancer cell line, and one hepatocellular carcinoma cell line. WT1 gene expression was detected in three of the four gastric cancer cell lines, all of the five colon cancer cell lines, 12 of the 15 lung cancer cell lines, two of the four breast cancer cell lines, the germ cell tumor cell line, the two ovarian cancer cell lines, the uterine cancer cell line, the thyroid cancer cell line, and the hepatocellular carcinoma cell line. Furthermore, when a gastric cancer cell line AZ-521, a lung cancer cell line OS3, and an ovarian cancer cell line TYK-nu were treated with WT1 antisense oligomers, the growth of these cells was significantly inhibited in association with a reduction in WT1 protein levels. Thus, there is indication that the WT1 gene plays an oncogenic role in the growth of several types of solid tumors.

It has been recently shown that the expression of high levels of the WT1 mRNA is associated with invasive breast cancers with poor patient prognosis (Miyoshi et al., 2002). However, the role of WT1 antisense molecules as possible treatments for breast cancer has not been investigated. As current cancer therapies have only limited therapeutic benefits, especially with regard to breast cancers, there exists a need for a treatment that is specific for different types of breast tumors.

### SUMMARY OF THE INVENTION

The present invention overcomes these and other defects in the art and demonstrates that antisense WT1 molecules are effective in inhibiting cancer cell growth in breast cancers expressing the Wilms' Tumor 1 (WT1) gene.

Thus, provided are methods for treating and/or preventing breast cancer. The invention also provides methods for diagnosing breast cancer and methods for screening for substances with activity against breast cancer.

In some embodiments, methods of inhibiting the growth of breast cancer cells expressing a WT1 gene product comprising contacting the cell with an amount of a WT1 antisense molecule effective to inhibit the growth of the breast cancer cell are provided.

An “effective amount” is defined here as an amount of a WT1 antisense molecule that will decrease, reduce, inhibit or otherwise abrogate the growth of a cancer cell, arrest-cell growth, induce apoptosis, inhibit metastasis, induce tumor necrosis, kill cells or induce cytotoxicity in cancer cells.

In some aspects of these embodiments, the cell may express one or more WT1 isoforms and/or one or more adverse oncogenes. The present invention contemplates that the growth of any breast cancer cell expressing a WT1 gene product may be inhibited. Thus, the breast cancer cell may be estrogen negative. Alternatively, the breast cancer cell may be estrogen positive.

In some embodiments, the WT1 antisense molecule may be a double stranded or single stranded DNA. In some specific embodiments, the DNA may be an oligonucleotide wherein the oligonucleotide may be 6 to about 50 bases in length comprising one or more modified bases. In other embodiments, the WT1 antisense molecule may be an RNA.

The antisense molecule may be produced from an expression vector encoding the WT1 antisense molecule under the control of a promoter active in the cell.

Any promoter active in a breast cancer cell may be used. However, some non-limiting examples are provided. For example, in some embodiments of the method, one may use a constitutive promoter, such as, a CMV promoter, an RSV promoter, or an SV40 promoter. In other embodiments, the promoter may be a tissue-specific promoter such as leptin gene promoter, IGF binding protein-3 promoter, adenomatous polyposis coli gene promoter. In yet other embodiments, the promoter may be an inducible promoter, for example, Tet-On system, Tet-Off system.

Expression vectors for the expression of antisense molecules as set forth herein are well known to one of skill in the art. In some embodiments, the expression vector may be a non-viral vector and/or a viral vector. Some examples of viral vectors include adenoviral vectors, retroviral vectors, herpesviral vectors, vaccinia viral vectors, adeno-associated viral vectors, lentiviral vectors or polyoma viral vectors.

In some embodiments of the method, the antisense molecule may hybridize to a WT1 transcript, a translation initiation site that may comprise 5′-GTCGGAGCCCATTTGCTG-3′ (SEQ ID NO: 1), a splice site, a genomic sequence, a transcription start site, an intron, an exon, and/or an intron-exon junction.

In other embodiments, the antisense molecule may be associated with one or more lipid molecules. In some specific aspects, the lipid may comprise at least one neutrally charged lipid. One example of a neutrally charged lipid is dioleoylphosphatidylcholine (DOPC). Other neutrally charged lipids known in the art may also be used. This includes lipids such as phosphatidylcholines, phosphatidylglycerols, and phosphatidylethanolamines.

In yet other aspects, the WT1 antisense molecule may be associated with more than one lipids wherein the lipids on a whole are neutrally charged. For example, the lipid component can comprise a mixture of positively and negatively charged lipids such that the overall charge of the lipid component is neutral.

In yet other embodiments, the antisense molecule may be encapsulated in a liposome. In some specific embodiments, the liposome may be comprised of at least one or more neutrally charged lipid molecules.

Another embodiment of the invention also provides methods of treating a subject having a breast cancer which express a Wilms' Tumor 1 (WT1) gene product, comprising administering to the subject an amount of an WT1 antisense molecule that is effective to treat the cancer.

The term “treat cancer” is defined as a decrease in cancer cell growth, reduction in cancer cell growth, inhibition or abrogation of growth of a cancer cell, cancer cell growth arrest, induction of apoptosis, killing of cancer cells, inhibition of metastasis, induction of tumor necrosis, and/or induction of cytotoxicity in cancer cells.

In such embodiments, the antisense molecule or formulations thereof may be administered to the tumor by intratumoral injection. In other embodiments, it may be administered to the tumor vasculature. In some other embodiments, it may be administered locally to the tumor. In yet other embodiments, it may be administered regionally. In other embodiments, it may be administered to the lymphatic system locally or regionally to the tumor.

In yet other embodiments, the antisense molecule or formulations thereof may be administered to the subject having such a tumor by systemic or parenteral methods of administration. This includes among others intravenous, intraarterial, intramuscular, intraperitoneal routes of administration.

The composition may advantageously be delivered to a human patient in a volume of 0.50-10.0 ml per dose, or in an amount of 5-100 mg antisense oligonucleotide per m² or 5-30 mg antisense oligonucleotide per m². Thus, one may administer 5 mg/m², 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², 19 mg/m², 20 mg/m², 21 mg/m², 22 mg/m², 23 mg/m², 24 mg/m², 25 mg/m², 26 mg/m², 27 mg/m², 28 mg/m², 29 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², or 100 mg/m² of a WT1 antisense oligonucleotide. Of course intermediate ranges are also contemplated as useful and this includes ranges such as 10.5 mg/m², 92 mg/m², and the like. As will be appreciated by one of skill in the art, the final dose of administration will be determined by a skilled physician depending on the disease status and individual suffering from the disease taking into effect factors such as age, sex, and the like. The composition may further be administered multiply, daily, weekly and/or monthly. As an example, it is contemplated that one particular therapeutic regimen the composition may be administered 3 times per week for 8 weeks.

It is also contemplated that the therapeutic methods may further comprise administering to a subject a second breast cancer therapy such as chemotherapy, radiation therapy, immunotherapy, hormonal therapy and/or gene therapy. Such methods are well known to a person of ordinary skill in the art and are also described elsewhere in the specification.

In some embodiments of the invention, the second breast cancer therapy may be provided to the subject prior to the WT1 antisense molecule. In other embodiments, the second breast cancer therapy may be provided to the subject after the WT1 antisense molecule. In yet other embodiments, the second breast cancer therapy may be provided to the subject at the same time as said WT1 antisense molecule.

The present invention also provides methods of predicting breast cancer progression in a subject having breast cancer that comprise obtaining a sample from the subject comprising breast cancer tumor cells and assessing expression of one or more isoforms of Wilms' Tumor 1 (WT1) gene product in the cells. In some embodiments, the assessing comprises measuring WT1 protein levels. In other embodiments, the assessing comprises measuring WT1 mRNA levels. In some embodiments, measuring these levels may comprise quantitative immunodetection methods and/or quantitative PCR. All these methods are known to a person of ordinary skill in the art and are also described elsewhere in the specification.

The present invention also provides methods of screening candidate substances for growth inhibitory activity against breast cancer comprising providing a cell that expresses one or more isoforms of the Wilms' Tumor 1 (WT1) gene product, contacting the cell with the candidate substance suspected of inhibiting WT1 and measuring the effect of the candidate substance on the cell wherein a decrease in the amount of WT1 gene product in the cell, as compared to a cell not treated with the candidate substance, indicates that the candidate substance has activity against breast cancer. The candidate substance may be a protein, a polypeptide, a nucleic acid and/or a small molecule pharmaceutical. In some embodiments of this method, the measuring may comprise determining the level of a WT1 gene product in the cell and/or determining the level of a WT1 gene transcript in the cell and/or determining the level of more than one WT1 gene product and/or determining the level of more than one WT1 transcript isoform and/or measuring the level of WT1 gene product in a cell not treated with the candidate substance.

“A” or “an” is defined herein to mean one or more than one. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Western blot analysis of WT1 expression in nuclear extracts of breast cancer cells. Nuclear mitotic apparatus protein (NUMA) was used as an internal control.

FIGS. 2A, 2B, 2C, and 2D. Growth inhibition of breast cancer cell lines by L-WT1. FIG. 2A. K562 cells—light bars: L-control; dark bars: L-WT1. FIG. 2B. MDA-MB-453 (□), and MCF-7 (Δ) cells treated with L-control oligos; MDA-MB-453(▪) and MCF-7 (▴) cells treated with L-WT1 oligos. FIG. 2C. Effect of 12 μM L-WT1 in 9 breast cancer cell lines. light bars: L-control; dark bars: L-WT1. FIG. 2D. Western blot of WT1 protein expression in MCF-7 and MDA-MB-453 cells exposed to L-WT1 and L-control oligos.

FIG. 3. Reduction in numbers of breast cancer cells by L-WT1. MCF-7 and MDA-MB-453 cells were treated with 12 μM L-WT1 or L-control oligos for 3 days and observed under light microscopy.

FIGS. 4A, 4B, and 4C. Expression of WT1 mRNA isoforms in breast cancer cell lines. ER-positive cell lines: 1: MCF-7; 2: BT-474; 3: T-47D; 4: MDA-MB-361. ER-negative cell lines: 5: SKBr-3; 6: MDA-MB-231; 7: MDA-MB-453; 8: BT-20; 9: MDA-MB-468, and 10: K562 leukemic cells. FIG. 4A. Results from a single round of RT-PCR analysis of total WT1 mRNA in breast cancer cell lines. FIG. 4B. Results from nested RT-PCR analysis of the KTS+ and the KTS− isoforms of WT1 mRNA. FIG. 4C. Results from nested RT-PCR analysis of all 4 isoforms of WT1 mRNA.

### DESCRIPTION OF ILLUSTRATIVE EMBODIMENTS

As mentioned above, breast cancer is the second most common form of cancer among women in the U.S., and the second leading cause of cancer deaths among women. While many therapies exist, these are either insufficient to eradicate the disease or are too toxic or both. Thus, there is a need to provide improved therapies and to better predict the progression of breast cancer.

### I. THE PRESENT INVENTION

The Wilms' Tumor 1 (WT1) gene modulates the expression of several genes involved in mammary glands. The inventors have identified a role for WT1 in the proliferation of breast cancer cells. The present invention provides a therapy that makes use of antisense oligonucleotides to reduce WT1 protein expression and induce growth inhibition of breast cancer cells. A particular method for delivering these antisense molecules is in association with lipids and in some embodiments via liposomes.

Some breast cancer cells are estrogen receptor (ER)-positive and some are ER-negative. While WT1 is expressed in higher levels in ER-positive cells, liposomal WT1 (L-WT1) is effective at inhibiting proliferation of breast cancer cells irrespective of their ER status. In addition, it is contemplated that the L-WT1 will be useful in inhibiting even those cells that have a high level of expression of adverse oncogenes such as EGFR, Her2/neu, and the mutant p53 protein. Thus, this technology holds great promise as a therapeutic agent for the treatment of cancer.

The present invention further contemplates the prediction of breast cancer progression in an individual having breast cancer by assessing expression of one or more isoforms of Wilms' Tumor 1 (WT1) gene product in said cells.

It also contemplates a method of screening a substance for its ability to suppress the WT1 protein expression in a cancer cell thus acting as a potential inhibitor of breast cancer. The invention, in its various embodiments, is described in greater detail below.

### II. WILMS' TUMOR GENE (WT1)

The chromosome 11p13 Wilms' Tumor susceptibility gene (WT1) appears to play a crucial role in regulating the proliferation and differentiation of nephroblasts and gonadal tissue. When present in the germline, specific heterozygous dominant-negative mutations are associated with severe abnormalities of renal and sexual differentiation, pointing to the essential role of WT1 for normal genitourinary development.

WT1 encodes a protein migrating around 50 kDa, which contains two domains with apparent functional properties: a C-terminal domain that consists of four Cys₂-His₂ zinc finger domains involved in DNA binding and an N-terminal proline/glutamine-rich transactivational domain. The zinc finger domains have a high degree of homology to the early growth response 1 and 2 products (Sukhatme et al., 1988; Joseph et al., 1988). The coding sequence is comprised of 10 exons, with each zinc finger encoded by an individual exon. Each of the four zinc finger domains is contained within a separate exon. The genomic structure of the zinc finger domains has been analyzed in which a small deletion has been detected (Haber et al., 1990). The analysis demonstrated that each zinc finger is separated from the next by a short intron.

Two alternative pre-mRNA splicing events give rise to four distinct transcripts or isoforms. Alternative splice I consists of 51 nucleotides, encoding 17 amino acids, including 5 serines and 1 threonine, potential sites of protein phosphorylation. The proline rich amino-terminus domain is encoded by the first exon alone, and the 51 nucleotides of alternative splice I compose exon 5. Splice I is inserted between the proline-rich amino terminus of the predicted protein and the first zinc finger domain.

Alternative splice II, results from the use of a variable splice donor site between exon 9 and 10, leading to the insertion of three amino acids, lysine-threonine-serine (commonly referred to as KTS), between third and fourth zinc finger. This insertion disrupts the critical spacing between these zinc fingers resulting in the loss of DNA binding to the consensus WT1 DNA-binding sequence (Wang et al., 1995).

The presence of two alternative splices in the WT1 trancript may reflect a degree of complexity in gene product function. The molecular mechanisms resulting in alternative mRNA splicing are poorly understood, but are thought to reflect both nucleotide sequence information contained in the splice junction, as well as cell type-specific regulatory factors (Breitbart et al., 1987).

Genetic evidence suggests that WT1 mutations, deletions, or imbalances among the different WT1 isoforms may alter the transcriptional-regulator function of WT1 leading to developmental abnormalities and possibly cancer (Klamt et al., 1998; Guan et al., 1998; Liu et al., 1999). High expression of WT1 has been correlated with poor prognosis and increased drug resistance in acute myeloid leukemia (Inoue et a., 1994), probably because increased WT1 expression can stimulate the proliferation and block the differentiation of leukemic cells (Yamagami et al., 1996). Therefore, WT1 seems to act as both a tumor suppressor gene and an oncogene in certain types of malignancies. Recently, two groups have reported that breast cancer cells also express WT1 protein, but they did not describe the function of WT1 in breast cancer cells (Silberstein et al., 1997; Loeb et al., 2001).

### III. ANTISENSE CONSTRUCTS

The term “antisense” is intended to refer to oligonucleotide or polynucleotide molecules complementary to a portion of a WT1 RNA, or the DNA's corresponding thereto. “Complementary” oligonucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

As used herein, the terms “complementary” or “antisense” mean oligonucleotides that are substantially complementary over their entire length and have very few base mismatches. For example, sequences of seven bases in length may be termed complementary when they have a complementary nucleotide for five or six positions out of seven. Naturally, sequences which are “completely complementary” will be sequences which are entirely complementary throughout their entire length and have no base mismatches.

Alternatively, the hybridizing segments may be shorter oligonucleotides. While all or part of the gene sequence may be employed in the context of antisense construction, it is important that the antisense when constructed binds/hybridizes the target sequence and does not face interference from other sequences that may be present in the gene sequence. Statistically, any sequence 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 6, 7, 8, 9, 10, 11, 12, 13, 14, 15, 16, 17, 18, 19, 20, 25, 30, 35, 40, 45 or 50 base pairs will be used. In the present invention, SEQ ID NO: 1 is the sequence of the WT1 antisense oligos targeted against the translation initiation site and SEQ ID NO: 2 is the sequence of the control oligos. One can readily determine whether a given antisense nucleic acid is effective in targeting of the corresponding host cell gene simply by testing the constructs in vitro to determine whether the endogenous gene's function is affected or whether the expression of related genes having complementary sequences is affected.

Targeting double-stranded (ds) DNA with oligonucleotides leads to triple-helix formation; targeting RNA will lead to double-helix formation. Antisense oligonucleotides, when introduced into a target cell, specifically bind to their target oligonucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

The intracellular concentration of monovalent cation is approximately 160 mM (10 mM Na⁺; 150 mM K⁺). The intracellular concentration of divalent cation is approximately 20 mM (18 mM Mg⁺⁺; 2 mM Ca⁺⁺). The intracellular protein concentration, which would serve to decrease the volume of hybridization and, therefore, increase the effective concentration of nucleic acid species, is 150 mg/ml. Constructs can be tested in vitro under conditions that mimic these in vivo conditions.

Antisense constructs may be designed to hybridize to a WT1 transcript, a translation initiation site, a splice site, a WT1 genomic sequence, a start site, an intron, an exon or an intron-exon junction.

Hybridization is a process by which two complementary nucleic acid strands, such as DNA and DNA, RNA and DNA or RNA and RNA, recognize and bind to each other and form a double stranded structure. Intracellular hybridization is the basis of antisense therapy. This involves the administration/delivery of an antisense nucleic acid to a cell where the antisense molecule finds its complementary target-nucleic acid, which may be either DNA or RNA, and hybridizes to it thereby preventing further transcription or translation of the target-nucleic acid. In a particular embodiment of the invention, it is contemplated that the most effective antisense constructs for the present invention will include regions complementary to portions of the mRNA start site. One can readily test such constructs simply by testing the constructs in vitro to determine whether levels of the target protein are affected. Similarly, detrimental non-specific inhibition of protein synthesis also can be measured by determining target cell viability in vitro. It is envisioned that hybridization of the antisense oligonucleotides of the present invention to the translation initiation site of mRNA will be the basis of the antisense-gene therapy aimed at WT1 mediated diseases. Intracellular hybridization will prevent the transcription of mRNA and thereby decrease the protein content in the cell to which the antisense oligonucleotide is administered.

Other sequences with lower degrees of homology also are contemplated. For example, an antisense construct which has limited regions of high homology, but also contains a non-homologous region (e.g., a ribozyme) could be designed. These molecules, though having less than 50% homology, would bind to target sequences under appropriate conditions.

As mentioned above, the oligonucleotides according to the present invention may encode a WT1 gene or a portion of that gene that is sufficient to effect antisense inhibition of expression of WT1 protein. These oligonucleotides may be derived from genomic DNA, i.e., cloned directly from the genome of a particular organism. In other embodiments, however, the oligonucleotides may be complementary DNA (cDNA). cDNA is DNA prepared using messenger RNA (mRNA) as template. Thus, a cDNA does not contain any interrupted coding sequences and usually contains almost exclusively the coding region(s) for the corresponding protein. In other embodiments, the antisense oligonucleotide may be produced synthetically.

It may be advantageous to combine portions of the genomic DNA with cDNA or synthetic sequences to generate specific constructs. For example, where an intron is desired in the ultimate construct, a genomic clone will need to be used. The cDNA or a synthesized oligonucleotide may provide more convenient restriction sites for the remaining portion of the construct and, therefore, would be used for the rest of the sequence.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al., 1993).

As an alternative to targeted antisense delivery, targeted ribozymes may be used. The term “ribozyme” refers to an RNA-based enzyme capable of targeting and cleaving particular base sequences in both DNA and RNA. Ribozymes can either be targeted directly to cells, in the form of RNA oligonucleotides incorporating ribozyme sequences, or introduced into the cell as an expression vector encoding the desired ribozymal RNA. Ribozymes may be used and applied in much the same way as described for antisense oligonucleotide. Ribozyme sequences also may be modified in much the same way as described for antisense oligonucleotide. For example, one could incorporate non-Watson-Crick bases, or make mixed RNA/DNA oligonucleotides, or modify the phosphodiester backbone.

Alternatively, the antisense oligo- or polynucleotides of the present invention may be provided as mRNA via transcription from expression constructs that carry nucleic acids encoding the oligonucleotides.

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques, as described in EP 266,032 incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotides may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid includes one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

### IV. GENETIC CONSTRUCTS

The nucleic acid segments of the present invention, regardless of the length of the coding sequence itself, may be combined with other DNA sequences, such as promoters, enhancers and polyadenylation signals. It will be important to employ a promoter that effectively directs the expression of the DNA segment in the cell type, organism, or even animal, chosen for expression. Throughout this application, the term “expression construct” is meant to include any type of genetic construct containing an antisense product in which part or all of the nucleic acid sequence is capable of being transcribed. Typical expression vectors include bacterial plasmids or phage, such as any of the pUC or Bluescript™ plasmid series or, as discussed further below, viral vectors adapted for use in eukaryotic cells.

A. Promoters

In particular embodiments, the antisense oligonucleotide or polynucleotide is part of an expression construct and is under the transcription control of a promoter. A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter employed to control the expression of a nucleic acid encoding the antisense oligonucleotides of this invention is not believed to be important, so long as it is capable of directing the expression of the antisense oligonucleotides in the targeted cell. Thus, where a human cell is targeted, it is preferable to position the nucleic acid coding the antisense oligonucleotide described in the invention adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus (RSV) long terminal repeat can be used to obtain high-level expression of the antisense oligonucleotides described and contemplated in the present invention. The use of other viral or mammalian cellular or bacterial phage promoters which are well-known in the art to achieve expression of an antisense oligonucleotide of interest is contemplated as well, provided that the levels of expression are sufficient for a given purpose.

Selection of a promoter that is regulated in response to specific physiologic or synthetic signals can permit inducible expression of the WT1 antisense oligonucleotide. For example, in the case where expression of a transgene or transgenes when a multicistronic vector is utilized, is toxic to the cells in which the vector is produced, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. Coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild-type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the patient, the gene expression would be constitutively on.

In some circumstances, it may be desirable to regulate expression of a transgene in a gene therapy vector. For example, different viral promoters with varying strengths of activity may be utilized depending on the level of expression desired. In mammalian cells, the CMV immediate early promoter is often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as leptin gene promoter (O'Neil et al., 2001), CDH13 (Toyooka et al., 2001), adenomatous polyposis coli (APC) gene promoter (Jin et al., 2001), IGF binding protein-3 promoter (IGFBP-3) (Walker et al., 2001) may be used to target gene expression in breast cancers.

By employing a promoter with well-known properties, the level and pattern of expression of an antisense oligonucleotide of interest can be optimized. Further, selection of a promoter that is regulated in response to specific physiologic signals can permit inducible expression of an antisense oligonucleotide. For example, a nucleic acid under control of the human PAI-1 promoter results in expression inducible by tumor necrosis factor. Tables 1 and 2 list several elements/promoters which may be employed, in the context of the present invention, to regulate the expression of antisense constructs. This list is not intended to be exhaustive of all the possible elements involved in the promotion of expression but, merely, to be exemplary thereof.

B. Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding an antisense oligonucleotide described in this invention in an expression construct (Table 1 and Table 2). Additionally any promoter/enhancer combination (as per the Eukaryotic Promoter Data Base EPDB) also could be used to drive expression of a nucleic acid according to the present invention. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
Other Promoter/Enhancer Elements

| Promoter/Enhancer | References |
|---|---|
| Immunoglobulin Heavy Chain | Banerji et al., 1983; Gilles et al., 1983; Grosschedl |
| | and Baltimore, 1985; Atchinson and Perry, 1986, |
| | 1987; Imler et al., 1987; Weinberger et al., 1988; |
| | Kiledjian et al., 1988; Porton et al., 1990 |
| Immunoglobulin Light Chain | Queen and Baltimore, 1983; Picard and Schaffner, |
| | 1984 |
| T-Cell Receptor | Luria et al., 1987, Winoto and Baltimore, 1989; |
| | Redondo et al., 1990 |
| HLA DQ α and DQ β | Sullivan and Peterlin, 1987 |
| β-Interferon | Goodbourn et al., 1986; Fujita et al., 1987; |
| | Goodbourn and Maniatis, 1985 |
| Interleukin-2 | Greene et al., 1989 |
| Interleukin-2 Receptor | Greene et al., 1989; Lin et al., 1990 |
| MHC Class II | Koch et al., 1989 |
| MHC Class II HLA-DRα | Sherman et al., 1989 |
| β-Actin | Kawamoto et al., 1988; Ng et al., 1989 |
| Muscle Creatine Kinase | Jaynes et al., 1988; Horlick and Benfield, 1989; |
| | Johnson et al., 1989a |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987, Tronche et al., 1989, 1990 |
| α-Fetoprotein | Godbout et al., 1988; Campere and Tilghman, |
| | 1989 |
| γ-Globin | Bodine and Ley, 1987; Perez-Stable and |
| | Constantini, 1990 |
| β-Globin | Trudel and Constantini, 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| a₁-antitrypsin | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh |
| | and Lockett, 1985; Firak and Subramanian, 1986; |
| | Herr and Clarke, 1986; Imbra and Karin, 1986; |
| | Kadesch and Berg, 1986; Wang and Calame, 1986; |
| | Ondek et al., 1987; Kuhl et al., 1987 Schaffner et |
| | al., 1988 |
| Polyoma | Swartzendruber and Lehman, 1975; Vasseur et al., |
| | 1980; Katinka et al., 1980, 1981; Tyndell et al., |
| | 1981; Dandolo et al., 1983; deVilliers et al., 1984; |
| | Hen et al., 1986; Satake et al., 1988; Campbell and |
| | Villarreal, 1988 |
| Retroviruses | Kriegler and Botchan, 1982, 1983; Levinson et al., |
| | 1982; Kriegler et al., 1983, 1984a, b, 1988; Bosze |
| | et al., 1986; Miksicek et al., 1986; Celander and |
| | Haseltine, 1987; Thiesen et al., 1988; Celander et |
| | al., 1988; Chol et al., 1988; Reisman and Rotter, |
| | 1989 |
| Papilloma Virus | Campo et al., 1983; Lusky et al., 1983; Spandidos |
| | and Wilkie, 1983; Spalholz et al., 1985; Lusky and |
| | Botchan, 1986; Cripe et al., 1987; Gloss et al., |
| | 1987; Hirochika et al., 1987, Stephens and |
| | Hentschel, 1987; Glu et al., 1988 |
| Hepatitis B Virus | Bulla and Siddiqui, 1986; Jameel and Siddiqui, |
| | 1986; Shaul and Ben-Levy, 1987; Spandau and |
| | Lee, 1988 |
| Human Immunodeficiency Virus | Muesing et al., 1987; Hauber and Cullan, 1988; |
| | Jakobovits et al., 1988; Feng and Holland, 1988; |
| | Takebe et al., 1988; Rowen et al., 1988; Berkhout |
| | et al., 1989; Laspia et al., 1989; Sharp and |
| | Marciniak, 1989; Braddock et al., 1989 |
| Cytomegalovirus | Weber et al., 1984; Boshart et al., 1985; Foecking |
| | and Hofstetter, 1986 |
| Gibbon Ape Leukemia Virus | Holbrook et al., 1987; Quinn et al., 1989 |

TABLE 2
Inducible Elements

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X |
| | poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |
| Insulin E Box | Glucose |

In certain embodiments of this invention, the delivery of a nucleic acid to a cell may be identified in vitro or in vivo by including a marker in the expression construct. The marker would result in an identifiable change to the transfected cell permitting easy identification of expression. Enzymes such as herpes simplex virus thymidine kinase (tk) (eukaryotic) or chloramphenicol acetyltransferase (CAT) (prokaryotic) may be employed.

C. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

### V. LIPID FORMULATIONS

In a particular embodiment of the invention, the antisense oligonucleotides and expression vectors may be associated with a lipid. An oligonucleotide associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid or lipid/oligonucleotide associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. An example is the lipid dioleoylphosphatidylcholine (DOPC).

Phospholipids may be used for preparing the liposomes according to the present invention and can carry a net positive charge, a net negative charge or are neutral. Diacetyl phosphate can be employed to confer a negative charge on the liposomes, and stearylamine can be used to confer a positive charge on the liposomes. The liposomes can be made of one or more phospholipids.

In a particular embodiment, the lipid material is comprised of a neutrally charged lipid. A neutrally charged lipid can comprise a lipid without a charge, a substantially. uncharged lipid or a lipid mixture with equal number of positive and negative charges.

In one aspect, the lipid component of the composition comprises a neutral lipid. In another aspect, the lipid material consists essentially of neutral lipids which is further defined as a lipid composition containing at least 70% of lipids without a charge. In other aspects, the lipid material may contain at least 80% to 90% of lipids without a charge. In yet other aspects, the lipid material may comprise about 90%, 95%, 96%, 97%, 98%, 99% or 100% lipids without a charge.

In specific aspects, the neutral lipid comprises a phosphatidylcholine, a phosphatidylglycerol, or a phosphatidylethanolamine. In a particular aspect, the phosphatidylcholine comprises DOPC.

In other aspects the lipid component comprises a substantially uncharged lipid. A substantially uncharged lipid is described herein as a lipid composition that is substantially free of anionic and cationic phospholipids and cholesterol. In yet other aspects the lipid component comprises a mixture of lipids to provide a substantially uncharged lipid. Thus, the lipid mixture may comprise negatively and positively charged lipids.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

Liposome-mediated oligonucleotide delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the lipid may be associated with a hemagglutinating virus (HVJ). This has been shown to facilitate fusion with the cell membrane and promote cell entry of liposome-encapsulated DNA (Kaneda et al., 1989). In other embodiments, the lipid may be complexed or employed in conjunction with nuclear non-histone chromosomal proteins (HMG-1) (Kato et al., 1991). In yet further embodiments, the lipid may be complexed or employed in conjunction with both HVJ and HMG-1. Such expression vectors have been successfully employed in transfer and expression of an oligonucleotide in vitro and in vivo and thus are applicable for the present invention. Where a bacterial promoter is employed in the DNA construct, it also will be desirable to include within the liposome an appropriate bacterial polymerase.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY—YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. A particular method of the invention describes the preparation of liposomes and is described below. Briefly, P-ethoxy oligonucleotides (also referred to as PE oligos) are dissolved in DMSO and the phospholipids (Avanti Polar Lipids, Alabaster, Ala.), such as for example the preferred neutral phospholipid dioleoylphosphatidylcholine (DOPC), is dissolved in tert-butanol. The lipid is then mixed with the antisense oligonucleotides. In the case of DOPC, the molar ratio of the lipid to the antisense oligos is 20:1. Tween 20 is added to the lipid:oligo mixture such that Tween 20 is 5% of the combined weight of the lipid and oligo. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the lipid with the oligo is 0.7-1.0 μm in diameter.

Alternatively liposomes can be prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures: the method of Bangham et al. (1965), the contents of which are incorporated herein by reference; the method of Gregoriadis (1979), the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated nucleic acid is removed by centrifugation at 29,000×g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentrations and stored at 4° C. until use.

P-ethoxy oligonucleotides, nucleases resistant analogues of phosphodiesters, are preferred because they are stable in serum. Neutral lipids are also preferred and specifically the lipid dioleoylphosphatidylchoine is preferred. However other lipids such as other phosphatidylcholines, phosphatidylglycerols, and phosphatidylethanolamines may also be useful. In yet another particular method described herein, the nuclease-resistant oligonucleotides and lipids are dissolved in DMSO and t-butanol respectively. The lipid is then mixed with the oligonucleotides in a molar ratio of between about 5:1 to about 100:1, and preferably in a ratio of 20:1. The preferred lipid:oligonucleotide ratio for P-ethoxy oligonucleotides and the lipid dioleoylphosphatidylchoine is 20:1. Tween 20 is then added to the mixture to obtain the liposomes. Excess t-butanol is added and the mixture is vortexed, frozen in an acetone/dry-ice bath, and then lyophilized overnight. The preparation is stored at −20° C. and may be used within one month of preparation. When required for use the lyophilized liposomal antisense oligonucleotides are reconstituted in 0.9% saline.

In an alternative embodiment, nuclease-resistant oligonucleotides are mixed with lipids in the presence of excess t-butanol. The mixture is vortexed before being frozen in an acetone/dry ice bath. The frozen mixture is then lyophilized and hydrated with Hepes-buffered saline (1 mM Hepes, 10 mM NaCl, pH 7.5) overnight, and then the liposomes are sonicated in a bath type sonicator for 10 to 15 min. The size of the liposomal-oligonucleotides typically ranges between 200-300 nm in diameter as determined by the submicron particle sizer autodilute model 370 (Nicomp, Santa Barbara, Calif.).

A pharmaceutical composition comprising the liposomes will usually include a sterile, pharmaceutically acceptable carrier or diluent, such as water or saline solution.

### VI. NON-LIPOSOMAL DELIVERY SYSTEMS

The delivery of antisense constructs of the present invention may also be accomplished using expression vectors which may be viral or non-viral in nature.

Retroviruses. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol, and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a WT1 antisense construct as described in this invention is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing an inserted DNA, together with the retroviral LTR and Ψ sequences, is introduced into this cell line (by calcium phosphate precipitation for example), the Ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Adenoviruses. Human adenoviruses are double-stranded DNA tumor viruses with genome sizes of approximate 36 kB. As a model system for eukaryotic gene expression, adenoviruses have been widely studied and well characterized, which makes them an attractive system for development of adenovirus as a gene transfer system. This group of viruses is easy to grow and manipulate, and they exhibit a broad host range in vitro and in vivo. In lytically infected cells, adenoviruses are capable of shutting off host protein synthesis, directing cellular machineries to synthesize large quantities of viral proteins, and producing copious amounts of virus.

The E1 region of the genome includes E1A and E1B which encode proteins responsible for transcription regulation of the viral genome, as well as a few cellular genes. E2 expression, including E2A and E2B, allows synthesis of viral replicative functions, e.g. DNA-binding protein, DNA polymerase, and a terminal protein that primes replication. E3 gene products prevent cytolysis by cytotoxic T cells and tumor necrosis factor and appear to be important for viral propagation. Functions associated .with the E4 proteins include DNA replication, late gene expression, and host cell shutoff. The late gene products include most of the virion capsid proteins, and these are expressed only after most of the processing of a single primary transcript from the major late promoter has occurred. The major late promoter (MLP) exhibits high efficiency during the late phase of the infection (Stratford-Perricaudet and Perricaudet, 1991).

A small portion of the viral genome appears to be required in cis adenovirus-derived vectors when used in connection with cell lines such as 293 cells. Ad5-transformed human embryonic kidney cell lines (Graham et al., 1977) have been developed to provide the essential viral proteins in trans.

Particular advantages of an adenovirus system for expressing and delivering the antisense oligonucleotides of this invention include (i) the structural stability of recombinant adenoviruses; (ii) the safety of adenoviral administration to humans; and (iii) lack of any known association of adenoviral infection with cancer or malignancies; (iv) the ability to obtain high titers of the recombinant virus; and (v) the high infectivity of adenovirus.

Further advantages of adenovirus vectors over retroviruses include the higher levels of gene expression. Additionally, adenovirus replication is independent of host gene replication, unlike retroviral sequences. Because adenovirus transforming genes in the E1 region can be readily deleted and still provide efficient expression vectors, oncogenic risk from adenovirus vectors is thought to be negligible (Grunhaus et al., 1992).

In general, adenovirus gene transfer systems are based upon recombinant, engineered adenovirus which is rendered replication-incompetent by deletion of a portion of its genome, such as E1, and yet still retains its competency for infection. Sequences encoding relatively large foreign proteins can be expressed when additional deletions are made in the adenovirus genome. Surprisingly persistent expression of transgenes following adenoviral infection has also been reported.

Other Viral Vectors as Expression Constructs. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) lentivirus, polyoma virus and herpes viruses may be employed. They offer several attractive features for various mammalian cells (Friedman et al., 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. (1991) introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Non-viral Methods. Several non-viral methods for the transfer of expression vectors into cultured mammalian cells also are contemplated in the present invention. These include calcium phosphate precipitation (Graham and van der Eb, 1973; Chen and Okayama, 1987; Rippe et al., 1990); DEAE-dextran (Gopal, 1985); electroporation (Tur-Kaspa et al., 1986; Potter et al., 1984); direct microinjection (Harland and Weintraub, 1985); cell sonication (Fecheimer et al., 1987); gene bombardment using high velocity microprojectiles (Yang et al., 1990); polycations; and receptor-mediated transfection (Wu and Wu, 1987; Wu and Wu, 1988). Some of these techniques may be successfully adapted for in vivo or ex vivo use.

In one embodiment of the invention, the expression construct may simply consist of naked recombinant vector. Transfer of the construct may be performed by any of the methods mentioned above which physically or chemically permeabilize the cell membrane. For example, Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding an WT1 antisense oligonucleotide construct may also be transferred in a similar manner in vivo.

Another embodiment of the invention for transferring a naked DNA expression vector into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ. DNA encoding a WT1 antisense oligonucleotide as described in this invention may be delivered via this method.

### VII. PHARMACEUTICALS

Where clinical application of liposomes containing antisense oligo- or polynucleotides or expression vectors is undertaken, it will be necessary to prepare the liposome complex as a pharmaceutical composition appropriate for the intended application. Generally, this will entail preparing a pharmaceutical composition that is essentially free of pyrogens, as well as any other impurities that could be harmful to humans or animals. One also will generally desire to employ appropriate buffers to render the complex stable and allow for uptake by target cells.

Aqueous compositions of the therapeutic composition of the present invention comprise an effective amount of the antisense expression vector encapsulated in a liposome as discussed above, further dispersed in pharmaceutically acceptable carrier or aqueous medium. Such compositions also are referred to as inocula. The phrases “pharmaceutically” or “pharmacologically acceptable” refer to compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

For human administration, preparations should meet sterility, pyrogenicity, general safety and purity standards as required by FDA Office of Biologics standards. The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains the therapeutic composition as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

A therapeutic composition can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical. Topical administration would be particularly advantageous for the treatment of skin cancers, to prevent chemotherapy-induced alopecia or other dermal hyperproliferative disorder. Alternatively, administration may be by orthotopic, intradernal subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, the preferred route is aerosol delivery to the lung. Volume of the aerosol is between about 0.01 ml and 0.5 ml. Similarly, a preferred method for treatment of colon-associated disease would be via enema. Volume of the enema is between about 1 ml and 100 ml.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting the dose include the physical and clinical state of the patient, the route of administration, the intended goal of treatment (alleviation of symptoms versus cure) and the potency, stability and toxicity of the particular therapeutic substance.

Administration of the therapeutic construct of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described treatments.

Depending on the particular cancer to be, administration of therapeutic compositions according to the present invention will be via any common route so long as the target tissue is available via that route. This includes oral, nasal, buccal, rectal, vaginal or topical. Topical administration would be particularly advantageous for treatment of skin cancers. Alternatively, administration will be by orthotopic, intradermal, subcutaneous, intramuscular, intraperitoneal or intravenous injection. Such compositions would normally be administered as pharmaceutically acceptable compositions that include physiologically acceptable carriers, buffers or other excipients.

The treatments may include various “unit doses.” Unit dose is defined as containing a predetermined-quantity of the therapeutic composition calculated to produce the desired responses in association with its administration, i.e., the appropriate route and treatment regimen. The quantity to be administered, and the particular route and formulation, are within the skill of those in the clinical arts. Also of importance is the subject to be treated, in particular, the state of the subject and the protection desired. A unit dose need not be administered as a single injection but may comprise continuous infusion over a set period of time.

According to the present invention, one may treat the cancer by directly injecting a tumor with the therapeutic composition of the present invention. Alternatively, the tumor may be infused or perfused with the antisense oligonucleotides using any suitable delivery vehicle. Local or regional administration, with respect to the tumor, also is contemplated. Finally, systemic administration may be performed. Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles or protein may advantageously be contacted by administering multiple injections to the tumor, spaced at approximately 1 cm intervals.

In certain embodiments, the tumor being treated may not, at least initially, be resectable. Treatments with therapeutic compositions may increase the resectability of the tumor due to shrinkage at the margins or by elimination of certain particularly invasive portions. Following treatments, resection may be possible. Additional viral or protein treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The preparation of more, or highly, concentrated solutions for direct injection is also contemplated, where the use of DMSO as solvent is envisioned to result in extremely rapid penetration, delivering high concentrations of the active agents to a small area.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

### VIII. COMBINATION CANCER THERAPY

To further enhance the efficacy of the therapy provided by the invention, combination therapies are contemplated. Thus, a second therapeutic agent in addition to a WT1 antisense oligonucleotide therapy may be used. The second therapeutic agent may be a chemotherapeutic agent, a radiotherapeutic agent, a gene therapeutic agent, a protein/peptide/polypeptide therapeutic agent, an immunotherapeutic agent, or a hormonal therapeutic agent. Such agents are well known in the art.

As set forth earlier an “effective amount” is defined as an amount of the WT1 antisense composition that can decrease, reduce, inhibit or otherwise abrogate the growth of a cancer cell, arrest-cell growth, induce apoptosis, inhibit metastasis, induce tumor necrosis, kill cells or induce cytotoxicity in cells.

The administration of the second therapeutic agent may precede or follow the therapy using an antisense construct by intervals ranging from minutes to days to weeks. In embodiments where the second therapeutic agent and an antisense construct encoding nucleic acid or protein product are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the second therapeutic agent and an antisense oligonucleotide will be required to achieve complete cancer cure. Various combinations may be employed, where the second therapeutic agent is “A” and the antisense oligonucleotide is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations also are contemplated. The exact dosages and regimens of each agent can be suitably altered by those of ordinary skill in the art.

Provided below is a description of some other agents effective in the treatment of cancer.

(i) Radiotherapeutic Agents

In some tumor cell lines, levels of antisense oligonucleotide were found to correlate to the sensitivity of cells to ionizing radiation, indicating that antisense therapy restores and/or enhances sensitivity of tumor cells to genotoxic agents. Therefore, additional therapy with radiotherapeutic agents and factors including radiation and waves that induce DNA damage for example, y-irradiation, X-rays, UV-irradiation, microwaves, electronic emissions, radioisotopes, and the like are contemplated. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

(ii) Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, clectrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

(iii) Chemotherapeutic Agents

Agents that damage DNA are chemotherapeutics. These can be, for example, agents that directly cross-link DNA, agents that intercalate into DNA, and agents that lead to chromosomal and mitotic aberrations by affecting nucleic acid synthesis. Agents that directly cross-link nucleic acids, specifically DNA, are envisaged and are exemplified by cisplatin, and other DNA alkylating agents. Agents that damage DNA also include compounds that interfere with DNA replication, mitosis, and chromosomal segregation.

Some examples of chemotherapeutic agents include antibiotic chemotherapeutics such as, Doxorubicin, Daunorubicin, Mitomycin (also known as mutamycin and/or mitomycin-C), Actinomycin D (Dactinomycin), Bleomycin, Plicomycin. Plant alkaloids such as Taxol, Vincristine, Vinblastine. Miscellaneous agents such as Cisplatin, VP16, Tumor Necrosis Factor. Alkylating Agents such as, Carmustine, Melphalan (also known as alkeran, L-phenylalanine mustard, phenylalanine mustard, L-PAM, or L-sarcolysin, is a phenylalanine derivative of nitrogen mustard), Cyclophosphamide, Chlorambucil, Busulfan (also known as myleran), Lomustine. And other agents for example, Cisplatin (CDDP), Carboplatin, Procarbazine, Mechlorethamine, Camptothecin, Ifosfamide, Nitrosurea, Etoposide (VP16), Tamoxifen, Raloxifene, Estrogen Receptor Binding Agents, Gemcitabien, Navelbine, Farnesyl-protein transferase inhibitors, Transplatinum, 5-Fluorouracil, and Methotrexate, Temazolomide (an aqueous form of DTIC), or any analog or derivative variant of the foregoing.

(iv) Immunotherapy

Immunotherapeutics may be used in conjunction with the therapy contemplated in the present invention. Immunotherapeutics, generally, rely on the use of immune effector cells and molecules to target and destroy cancer cells. The immune effector may be, for example, another antibody specific for some other marker on the surface of a tumor cell. This antibody in itself may serve as an effector of therapy or it may recruit other cells to actually effect cell killing. This antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target.

In one aspect the immunotherapy can be used to target a tumor cell. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. Alternate immune stimulating molecules also exist including: cytokines such as IL-2, IL-4, IL-12, GM-CSF, gamma-IFN, chemokines such as MIP-1, MCP-1, IL-8 and growth factors such as FLT3 ligand.

(a) Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

(b) Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993).

(c) Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro.

(v) Gene Therapy

The present invention contemplates the use of a variety of different therapeutic transgenes in combination with the antisense therapy of the present invention. For example, genes encoding a tumor suppressor, an inhibitor of apoptosis, a cell cycle regulatory gene, a toxin, a cytokine, a ribosome inhibitory protein and interferons are contemplated as suitable genes that potentiate the inhibition of cancer cell growth according to the present invention.

(a) Tumor Suppressors

The tumor suppressors function to inhibit excessive cellular proliferation. The inactivation of these genes destroys their inhibitory activity, resulting in unregulated proliferation. It is contemplated that the antisense oligonucleotide may be attached to antibodies that recognize mutant tumor suppressors or wild-type tumor suppressors. Alternatively, an antisense construct may be linked to all or part of the tumor suppressor. Exemplary tumor suppressors are p53, p16 and C—CAM which are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors. The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16 gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Okamoto et al., 1994; Nobori et al., 1995; Orlow et al., 1994). Restoration of wild-type p16^{INK4} function by transfection with a plasmid expression vector reduced colony formation by some human cancer cell lines (Okamoto, 1994).

Other genes that may be employed according to the present invention include Rb, APC, mda-7, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

(b) Regulators of Programmed Cell Death

Apoptosis, or programmed cell death, is an essential process for normal embryonic development, maintaining homeostasis in adult tissues, and suppressing carcinogenesis (Kerr et al., 1972). The Bc1-2 family of proteins and ICE-like proteases have been demonstrated to be important regulators and effectors of apoptosis in other systems. The Bc1-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bc1-2 protein now is recognized to be a member of a family of related proteins, which can be categorized as death agonists or death antagonists.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells (Marsters et al., 1999).

Subsequent to its discovery, it was shown that Bc1-2 acts to suppress cell death triggered by a variety of stimuli. Also, it now is apparent that there is a family of Bc1-2 cell death regulatory proteins which share in common structural and sequence homologies. These different family members have been shown to either possess similar functions to Bc1-2 (e.g., Bc1_{XL}, Bc1_{W}, Bc1_{S}, Mc1-1, A1, Bf1-1) or counteract Bc1-2 function and promote cell death (e.g., Bax, Bak, Bik, Bim, Bid, Bad, Harakiri). It is contemplated that any of these polypeptides, including TRAIL, or any other polypeptides that induce or promote of apoptosis, may be operatively linked to an antisense construct, or that an antibody recognizing any of these polypeptides may also be attached to an antisense construct.

It will be appreciated by those of skill in the art that monoclonal or polyclonal antibodies specific for proteins that are preferentially expressed in metastatic or nonmetastatic cancer will have utilities in several types of applications. These may include the production of diagnostic kits for use in detecting or diagnosing human cancer. An alternative use would be to link such antibodies to therapeutic agents, such as chemotherapeutic agents, followed by administration to individuals with cancer, thereby selectively targeting the cancer cells for destruction. The skilled practitioner will realize that such uses are within the scope of the present invention.

(c) Interferons

Other classes of genes that are contemplated to be inserted into the vectors of the present invention include interferons, interleukins and cytokines. Inteferon-α, interferon-β, interferon-γ, interleukin 1 (IL-1), IL-2, IL-3, IL-4, IL-5, IL-6, IL-7, IL-8, IL-9, IL-10, IL-11, IL-12, IL-13, IL-14, IL-15, angiostatin, thrombospondin, endostatin, METH-1, METH-2, Flk2/Flt3 ligand, GM-CSF, G-CSF, M-CSF, and tumor necrosis factor (TNF).

(d) Cell Cycle Regulatory Genes

In another embodiment, the present invention utilizes an isolated nucleic acid segment comprising a cell cycle regulatory gene operatively linked to an antisense oligonucleotide of the present invention; transferring the nucleic acid segment into a cancer cell to obtain a transfected cell; and maintaining the cancer cell under conditions effective to express the cell cycle regulatory gene; wherein expression of the cell cycle regulatory gene inhibits proliferation of the cancer cell. In the practice of the method, the cell cycle regulatory gene operatively linked to an antisense oligonucleotide may comprise a liposomal or a non-liposomal vector. In the present invention, it comprises a liposomal vector. Further, the cell cycle regulatory gene may preferably encode Rb, p53, cell cycle dependent kinase, CDK kinase, cyclin or a constitutively active Rb gene product, or an antisense RNA.

(e) Toxin Encoding Genes

In another embodiment, the present invention may be described as a method of inhibiting tumor cell growth comprising the steps of: obtaining an isolated nucleic acid segment comprising a toxin encoding gene. The genes may encode TNFα, gelonin, ricin A Chain, Pseudomonas exotoxin, diphtheria toxin, mitogillin, saporin, ribosome inhibitory protein.

(f) Oncogenes

Oncogenes are considered to be genes that, when mutated or activated, sponsor the development of cancer. Therapeutic intervention involves the inhibition of these gene products. For example, one may provide antisense or ribozymes which inhibit the transcription, processing or translation of an oncogene. Alternatively, single chain antibodies that encode products bind to and inhibit the oncogene can be utilized. Table 3 provides a list of suitable oncogene targets.

TABLE 3

| | | | |
|---|---|---|---|
| Gene | Source | Human Disease | Function |

| | | |
|---|---|---|
| Growth Factors | | |
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma | PDGF B |
| | virus | |

Receptor Tyrosine Kinases

| | | | |
|---|---|---|---|
| ERBB/HER | Avian | Amplified, deleted | EGF/TGF-α/ |
| | erythroblastosis | squamous cell | Amphiregulin/ |
| | virus; ALV | cancer; | Hetacellulin |
| | promoter | glioblastoma | receptor |
| | insertion; | | |
| | amplified | | |
| | human tumors | | |
| ERBB-2/NEU/HER- | Transfected from rat | Amplified breast, | Regulated by NDF/ |
| 2 | Glioblastomas | ovarian, gastric | Heregulin and |
| | | cancers | EGF-Related factors |
| FMS | SM feline sarcoma | | CSF-1 receptor |
| | virus | | |
| KIT | HZ feline sarcoma | | MGF/Steel receptor |
| | virus | | Hematopoieis |
| TRK | Transfection from | | NGF (nerve growth |
| | human colon | | Factor) receptor |
| | cancer | | |
| MET | Transfection from | | Scatter factor/HGF |
| | human | | Receptor |
| | osteosarcoma | | |
| RET | Translocations and | Sporadic thyroid | Orphan receptor Tyr |
| | point mutations | cancer; | Kinase |
| | | familial medullary | |
| | | thyroid cancer; | |
| | | multiple endocrine | |
| | | neoplasias 2A and | |
| | | 2B | |
| ROS | URII avian sarcoma | | Orphan receptor Tyr |
| | Virus | | Kinase |
| PDGF receptor | Translocation | Chronic | TEL(ETS-like |
| | | Myelomonocytic | transcription |
| | | Leukemia | factor)/ |
| | | | PDGF receptor |
| | | | gene |
| | | | Fusion |
| TGF-β receptor | | Colon carcinoma | |
| | | mismatch mutation | |
| | | target | |

NONRECEPTOR TYROSINE
KINASES

| | | | |
|---|---|---|---|
| ABI. | Abelson Mul. V | Chronic | Interact with RB, |
| | | myelogenous | RNA |
| | | leukemia | polymerase, CRK, |
| | | translocation | CBL |
| | | with BCR | |
| FPS/FES | Avian Fujinami | | |
| | SV; GA | | |
| | FeSV | | |
| LCK | Mul. V (murine | | Src family; T-cell |
| | leukemia | | signaling; interacts |
| | virus) promoter | | CD4/CD8 T-cells |
| | insertion | | |
| SRC | Avian Rous | | Membrane- |
| | sarcoma | | associated Tyr |
| | Virus | | kinase with |
| | | | signaling function; |
| | | | activated by |
| | | | receptor kinases |
| YES | Avian Y73 virus | | Src family; |
| | | | signaling |

SER/THR PROTEIN KINASES

| | | |
|---|---|---|
| AKT | AKT8 murine | Regulated by |
| | retrovirus | PJ(3)K?; |
| | | regulate 70-kd S6 |
| | | k? |
| MOS | Moloney murine SV | GVBD; cystostatic |
| | | factor; MAP |
| | | kinase |
| | | kinase |
| PIM-1 | Promoter insertion | |
| | Mouse | |
| RAF/MIL | 3611 murine SV; | Signaling in RAS |
| | MH2 | Pathway |
| | avian SV | |

MISCELLANEOUS CELL SURFACE

| | | | |
|---|---|---|---|
| APC | Tumor suppressor | Colon cancer | Interacts with |
| | | | catenins |
| DCC | Tumor suppressor | Colon cancer | CAM domains |
| E-cadherin | Candidate tumor | Breast cancer | Extracellular |
| | Suppressor | | homotypic |
| | | | binding; |
| | | | intracellular |
| | | | interacts with |
| | | | catenins |
| PTC/NBCCS | Tumor suppressor | Nevoid basal cell | 12 transmembrane |
| | and | cancer | domain; signals |
| | Drosophilia | syndrome (Gorline | through Gli |
| | homology | syndrome) | homogue |
| | | | CI to antagonize |
| | | | hedgehog pathway |
| TAN-1 Notch | Translocation | T-ALI. | Signaling? |
| homologue | | | |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | Phosphorylated |
| | | | RING |
| | | | finger interact Abl |
| CRK | CT1010 ASV | | Adapted SH2/SH3 |
| | | | interact Abl |
| DPC4 | Tumor suppressor | Pancreatic cancer | TGF-β-related |
| | | | signaling |
| | | | Pathway |
| MAS | Transfection and | | Possible angiotensin |
| | Tumorigenicity | | Receptor |
| NCK | | | Adaptor SH2/SH3 |

GUANINE NUCLEOTIDE EXCHANGERS AND BINDING
PROTEINS

| | | | |
|---|---|---|---|
| BCR | | Translocated with | Exchanger; protein |
| | | ABL | Kinase |
| | | in CML | |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey-Kirsten, N- | HaRat SV; Ki | Point mutations in | Signal cascade |
| RAS | RaSV; | many | |
| | Balb-MoMuSV; | human tumors | |
| | Transfection | | |
| VAV | Transfection | | S112/S113; |
| | | | exchanger |

NUCLEAR PROTEINS AND TRANSCRIPTION FACTORS

| | | | |
|---|---|---|---|
| BRCA1 | Heritable suppressor | Mammary | Localization |
| | | cancer/ovarian | unsettled |
| | | cancer | |
| BRCA2 | Heritable suppressor | Mammary cancer | Function unknown |
| ERBA | Avian | | thyroid hormone |
| | erythroblastosis | | receptor |
| | Virus | | (transcription) |
| ETS | Avian E26 virus | | DNA binding |
| EVII | MuLV promotor | AML | Transcription factor |
| | Insertion | | |
| FOS | FBI/FBR murine | | 1 transcription |
| | osteosarcoma | | factor |
| | viruses | | with c-JUN |
| GLI | Amplified glioma | Glioma | Zinc finger; cubitus |
| | | | interrupts |
| | | | homologue |
| | | | is in hedgehog |
| | | | signaling pathway; |
| | | | inhibitory link |
| | | | PTC |
| | | | and hedgehog |
| HMGI/LIM | Translocation | Lipoma | Gene fusions high |
| | t(3:12) | | mobility group |
| | t(12:15) | | HMGI-C (XT |
| | | | hook) |
| | | | and transcription |
| | | | factor |
| | | | LIM or acidic |
| | | | domain |
| JUN | ASV-17 | | Transcription factor |
| | | | AP-1 with FOS |
| MLL/VHRX + | Translocation/fusion | Acute myeloid | Gene fusion of |
| ELI/MEN | ELL with MLL | leukemia | DNA- |
| | Trithorax-like gene | | binding and methyl |
| | | | transferase MLL |
| | | | with |
| | | | ELI RNA pol II |
| | | | elongation factor |
| MYB | Avian | | DNA binding |
| | myeloblastosis | | |
| | Virus | | |
| MYC | Avian MC29; | Burkitt's lymphoma | DNA binding with |
| | Translocation B- | | MAX partner; |
| | cell | | cyclin |
| | Lymphomas; | | regulation; interact |
| | promoter | | RB?; regulate |
| | Insertion avian | | apoptosis? |
| | leukosis | | |
| | Virus | | |
| N-MYC | Amplified | Neuroblastoma | |
| L-MYC | | Lung cancer | |
| REL | Avian | | NF-κB family |
| | | | transcription factor |
| | Retriculoendotheliosis | | |
| | Virus | | |
| SKI | Avian SKV770 | | Transcription factor |
| | Retrovirus | | |
| VHL | Heritable suppressor | Von Hippel-Landau | Negative regulator |
| | | syndrome | or |
| | | | elongin; |
| | | | transcriptional |
| | | | elongation |
| | | | complex |
| WT-1 | | Wilms' tumor | Transcription factor |

CELL CYCLE/DNA DAMAGE
RESPONSE

| | | | |
|---|---|---|---|
| ATM | Hereditary disorder | Ataxia- | Protein/lipid kinase |
| | | telangiectasia | homology; DNA |
| | | | damage response |
| | | | upstream in P53 |
| | | | pathway |
| BCL-2 | Translocation | Follicular | Apoptosis |
| | | lymphoma | |
| FACC | Point mutation | Fanconi's anemia | |
| | | group | |
| | | C (predisposition | |
| | | leukemia | |
| MDA-7 | Fragile site 3p14.2 | Lung carcinoma | Histidine triad- |
| | | | related |
| | | | diadenosine |
| | | | 5′, 3′′′′- |
| | | | tetraphosphate |
| | | | asymmetric |
| | | | hydrolase |
| hMLI/MutL | | HNPCC | Mismatch repair; |
| | | | MutL |
| | | | Homologue |
| hMSH2/MutS | | HNPCC | Mismatch repair; |
| | | | MutS |
| | | | Homologue |
| hPMS1 | | HNPCC | Mismatch repair; |
| | | | MutL |
| | | | Homologue |
| hPMS2 | | HNPCC | Mismatch repair; |
| | | | MutL |
| | | | Homologue |
| INK4/MTS1 | Adjacent INK-4B at | Candidate MTS1 | p16 CDK inhibitor |
| | 9p21; CDK | suppressor and | |
| | complexes | MLM | |
| | | melanoma gene | |
| INK4B/MTS2 | | Candidate | p15 CDK inhibitor |
| | | suppressor | |
| MDM-2 | Amplified | Sarcoma | Negative regulator |
| | | | p53 |
| p53 | Association with | Mutated >50% | Transcription factor; |
| | SV40 | human | checkpoint control; |
| | T antigen | tumors, including | apoptosis |
| | | hereditary Li- | |
| | | Fraumeni | |
| | | syndrome | |
| PRAD1/BCL1 | Translocation with | Parathyroid | Cyclin D |
| | Parathyroid | adenoma; | |
| | hormone | B-CLL | |
| | or IgG | | |
| RB | Hereditary | Retinoblastoma; | Interact cyclin/cdk; |
| | Retinoblastoma; | osteosarcoma; | regulate E2F |
| | Association with | breast | transcription factor |
| | many | cancer; other | |
| | DNA virus tumor | sporadic | |
| | Antigens | cancers | |
| XPA | | xeroderma | Excision repair; |
| | | pigmentosum; skin | photo- |
| | | cancer | product |
| | | predisposition | recognition; |
| | | | zinc finger |

(g) Other Agents

It is contemplated that other agents may be used in combination with the present invention to improve the therapeutic efficacy of treatment. One form of therapy for use in conjunction with chemotherapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radio frequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention. The use of hormones may be employed in the treatment of certain cancers such as breast, prostate, ovarian, or cervical cancer to lower the level or block the effects of certain hormones such as testosterone or estrogen and this often reduces the risk of metastases.

The terms “contacted” and “exposed,” when applied to a cell, are used herein to describe the process by which a therapeutic construct or protein and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### IX. PROGNOSTIC APPLICATIONS

As described earlier, the WT1 mRNA can be spliced in two different ways leading to the expression of at least four predominant isoforms (Haber et al., 1991). One splicing inserts or removes 17 amino acids in exon 5; the other splicing inserts or removes the 3-amino-acid Lys-Thr-Ser (KTS) in exon 9, located between zinc fingers 3 and 4 (Lee et al., 2001; Wang et al., 1995). All of the resulting WT1 isoforms can positively or negatively regulate gene expression (Klamt et al., 1998). Throughout the application, reference to WT1 will encompass its various isoforms.

The WT1 splicing isoforms have different biological activities (Lee et al., 2001). Of the two major splicing products encoded by WT1, the −KTS isoforms have transactivational properties in some genes that are involved in cell growth and differentiation, whereas the +KTS isoforms have a potential role in RNA processing (Lee et al., 2001). Exon 5 may function as a repressor domain or as an activator domain, depending on which proteins are interacting with WT1 (Richard et al., 2001).

WT1 mRNA is readily detected by Northern (RNA) blot in most Wilms tumor, as well as normal fet al kidney tissue (Haber et al., 1990). With the more sensitive RNA PCR technique, alternatively spliced WT1 transcripts can be easily demonstrated in all tissues. However, PCR analysis can only provide an approximate ratio of the various RNA species and due to the positions and sizes of two alternative splices, it cannot be used to distinguish various splicing combinations.

To determine the existence and relative abundance of various forms of the WT1 transcript, an RNase protection assay has been developed which is capable of differentiating each form based on a protected fragment of distinctive length (Haber et al., 1991). The functional role of each WT1 isoform in breast cancer cells such as cell proliferation, sensitivity to estrogens and anti-estrogens, sensitivity to apoptotic and chemotherapeutic stimuli may enable one to determine whether a patient's breast tumor has high expression of a certain WT1 isoform, and may potentially be able to predict what kind of therapy the breast tumor will respond to. Techniques such as RT-PCR or RNase protection assay may be used to determine the level of expression of a certain WT1 isoform.

The present invention further contemplates that the evaluation of the expression level of one or more isoforms of WT1 gene product in a cancer cell will be useful to effectively predict the efficacy of a cancer therapeutic regimen, to determine whether the patient's cancer will be responsive to a particular cancer therapeutic regimen by analyzing the cancer tissues or cancer cells of a patient and to monitor the progression of breast cancer in a patient. The method of the present invention will involve obtaining a sample from said subject comprising breast cancer cells and assessing expression of one or more isoforms of Wilms' Tumor 1 (WT1) gene product in said cells.

The present invention's prognostic method therefore allows the determination of the need for specific cancer therapeutic regimens based on the expression of WT1 in an individual patient.

The expression levels of WT1 protein will also be useful in monitoring the effectiveness of a treatment regimen, such as that of the present invention, alone or in conjunction with other cancer therapies as described above. Again, in such a situation the level of expression of WT1 protein will be used to effectively determine and adjust the dosage of a radiation and/or chemotherapeutic combination regimen. In any event, the methods of the present invention will assist physicians in determining optimal treatment courses and doses for individuals with different tumors of varying malignancy based on the levels of expression of WT1 proteins in such tumors.

As described herein, the amount of a WT1 polypeptide/protein and/or mRNA present within a biological sample or specimen, such as a tissue, a cell(s), blood or serum or plasma sample, any other biological fluid, a biopsy, needle biopsy cores, surgical resection samples, lymph node tissue, or any other clinical sample may be determined by means of a molecular biological assay to determine the level of a nucleic acid that encodes such a polypeptide, or by means of a polypeptide/protein detection assay such as a western blot, northern blot (to quantitate RNA), RNA-PCR or even by means of an immunoassay may be detected and measured or quantified. Such detection and measuring/quantification methods may be used to measure WT1 protein levels or WT1 mRNA levels and such methods are known to one of skill in the art.

In certain embodiments, nucleic acids or polypeptides would be extracted from these samples. Some embodiments would utilize kits containing pre-selected primer pairs or hybridization probes, such as an antisense construct of the present invention. Antibodies may also be used for this purpose. The amplified nucleic acids or polypeptide would be tested for the presence of a WT1 polypeptide/protein and/or mRNA by any of the detection methods described later in the description or other suitable methods known in the art.

In other embodiments, sample/specimen extracts containing a WT1 polypeptide/protein and/or mRNA would be extracted from a sample and subjected to an immunoassay. Immunoassays of tissue sections are also possible. Immunoassays that are contemplated useful are well known to one of skill in the art. Kits containing the antibodies to WT1 polypeptides would be useful.

In terms of analyzing tissue samples, irrespective of the manner in which the level of a WT1 polypeptide/protein and/or mRNA is determined, the prognostic evaluation may generally require the amount of the WT1 product in the tissue sample to be compared to the amount in normal cells, in other patients and/or amounts at an earlier stage of treatment of the same patient. Comparing the varying levels will allow the characteristics of the particular cancer to be more precisely defined and therefore allow for prescribing a tailor made cancer treatment regimen to a patient.

Thus, it is contemplated that the levels of a WT1 polypeptide/protein and/or mRNA detected would be compared with statistically valid groups of metastatic, non-metastatic malignant, benign or normal tissue samples; and/or with earlier WT1 levels in the same patient. The diagnosis and prognosis of the individual patient would be determined by comparison with such groups.

If desired, the cancer prognostic methods of the present invention may be readily combined with other methods in order to provide an even more reliable indication of prognosis. Various markers of cancer have been proposed to be correlated with metastasis and malignancy. They are generally classified as cytological, protein or nucleic acid markers. Any one or more of such methods may thus be combined with those of this invention in order to provide a multi-marker prognostic test. Some examples of tumor markers specific to breast include p27, cyclin E, carcinoembryonic antigen (CEA), mucin associated antigen, tumor polypeptide antigen and breast cancer specific antigen.

Combination of the present techniques with one or more other diagnostic or prognostic techniques or markers is certainly contemplated. In that many cancers are multifactorial, the use of more than one method or marker is often highly desirable.

A. Prognostic Kits

The materials and reagents required for detecting the levels of expression of a WT1 polypeptide/protein and/or mRNA in a cancer cell in a biological sample may be assembled together in a kit.

One set of kits are designed to detect the levels of expression of a WT1 nucleic acid. Such kits of the invention will generally comprise one or more preselected primers or probes specific for WT1. The antisense constructs of the present invention may be used as hybridization probes or primers. Preferably, the kits will comprise, in suitable container means, one or more nucleic acid probes or primers and means for detecting nucleic acids. In certain embodiments, such as in kits for use in Northern blotting, the means for detecting the nucleic acids may be a label, such as a radiolabel, that is linked to a nucleic acid probe itself.

Preferred kits are those suitable for use in PCR™ which is described later in the specification. In PCR™ kits, two primers will preferably be provided that have sequences from, and that hybridize to, spatially distinct regions of the WT1 gene. Preferred pairs of primers for amplifying nucleic acids are selected to amplify the sequences specified herein. Also included in PCRTM kits may be enzymes suitable for amplifying nucleic acids, including various polymerases (RT, Taq, etc.), deoxynucleotides and buffers to provide the necessary reaction mixture for amplification.

In each case, the kits will preferably comprise distinct containers for each individual reagent and enzyme, as well as for each cancer probe or primer pair. Each biological agent will generally be suitable aliquoted in their respective containers.

The container means of the kits will generally include at least one vial or test tube. Flasks, bottles and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions may be provided with the kit.

In further embodiments, the invention provides immunological kits for use in detecting the levels of expression of WT1 in biological samples, e.g., in cancer cells. Such kits will generally comprise one or more antibodies that have immunospecificity for WT1 proteins or peptides.

As the anti-WT1 antibodies may be employed to detect WT1 proteins or peptides and their levels, both of such components may be provided in the kit. The immunodetection kits will thus comprise, in suitable container means, a WT1 protein or peptide, or a first antibody that binds to such a protein or peptide, and an immunodetection reagent.

In other embodiments, it is contemplated that the antibodies will be those that bind to the WT1 antigenic epitopes. MAbs are readily prepared and will often be preferred. Where proteins or peptides are provided, it is generally preferred that they be highly purified.

In certain embodiments, the WT1 protein or peptide, or the first antibody that binds to the WT1 protein or peptide may be bound to a solid support, such as a column matrix or well of a microtitre plate.

The immunodetection reagents of the kit may take any one of a variety of forms, including those detectable labels that are associated with, or linked to, the given antibody or antigen itself. Detectable labels that are associated with or attached to a secondary binding ligand are also contemplated. Exemplary secondary ligands are those secondary antibodies that have binding affinity for the first antibody or antigen.

Further suitable immunodetection reagents for use in the present kits include the two-component reagent that comprises a secondary antibody that has binding affinity for the first antibody or antigen (generally anti-WT1) along with a third antibody that has binding affinity for the second antibody, wherein the third antibody is linked to a detectable label.

As noted above in the discussion of antibody conjugates, a number of exemplary labels are known in the art and all such labels may be employed in connection with the present invention. Radiolabels, nuclear magnetic spin-resonance isotopes, fluorescent labels and enzyme tags capable of generating a colored product upon contact with an appropriate substrate are suitable examples.

The kits may contain antibody-label conjugates either in fully conjugated form, in the form of intermediates, or as separate moieties to be conjugated by the user of the kit.

The kits may further comprise a suitably aliquoted composition of a WT1 antigen whether labeled or unlabeled, as may be used to prepare a standard curve for a detection assay.

The kits of the invention, regardless of type, will generally comprise one or more containers into which the biological agents are placed and, preferably, suitable aliquoted. The components of the kits may be packaged either in aqueous media or in lyophilized form.

The immunodetection kits of the invention, may additionally contain one or more of a variety of other cancer marker antibodies or antigens, if so desired. Such kits could thus provide a panel of cancer markers, as may be better used in testing a variety of patients. By way of example, such additional markers could include, other tumor markers such as breast cancer antigen CA15-3, p53, BR 27.29, HER-2/neu, BRCA-1, and BRCA-2. The container means of the kits will generally include at least one vial, test tube, flask, bottle, or even syringe or other container means, into which the antibody or antigen may be placed, and preferably, suitably aliquoted. Where a second or third binding ligand or additional component is provided, the kit will also generally contain a second, third or other additional container into which this ligand or component may be placed.

The kits of the present invention will also typically include a means for containing the antisense composition, and any other reagent containers in close confinement for commercial sale. Such containers may include injection or blow-molded plastic containers into which the desired vials are retained.

### X. SCREENING ASSAYS

The present invention contemplates the screening of compounds for abilities to affect expression or function of isoforms of WT1. Particularly preferred compounds will be those useful in inhibiting the expression of WT1, thus inhibiting the growth of breast cancer cells. In the screening assays of the present invention, the candidate substance may first be screened for basic activity—e.g., binding to a target molecule—and then tested for its ability to inhibit expression, at the cellular, tissue or whole animal level.

The present invention provides methods of screening for candidate substances that show activity against breast cancer. In one embodiment, the present invention is directed to a method of:

(i) providing a cell that expresses one or more isoforms of the Wilms' Tumor 1 (WT1) gene product;

(ii) contacting the cell with the candidate substance suspected of inhibiting WT1; and

(iii) measuring the effect of the candidate substance on the cell.

The candidate substance may be a protein, a nucleic acid or a small molecule pharmaceutical. As a result of measurement, a decrease in the amount of one or more WT1 isoform gene products or gene transcripts in said cell, as compared to a cell not treated with said candidate substance, indicates that said candidate substance has activity against breast cancer.

In still yet other embodiments, one would look at the effect of a candidate substance on the expression of WT1. This can be done by examining mRNA expression, although the clinical results could be insufficient. A more direct way of assessing expression is by directly examining protein levels, for example, through Western blot or ELISA. An inhibitor according to the present invention may be one which exerts an inhibitory effect on the expression or function of WT1.

As used herein, the term “candidate substance” refers to any molecule that may inhibit growth of cancer cells. The candidate substance may be a protein or fragment thereof, a small molecule inhibitor, or even a nucleic acid molecule. It may prove to be the case that the most useful pharmacological compounds will be compounds that are structurally related to compounds which interact naturally with WT1. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like a WT1, and then design a molecule for its ability to interact with WT1. Alternatively, one could design a partially functional fragment of a WT1 (binding but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of hypertrophic response.

The candidate substance suspected of inhibiting WT1 expression may be an antisense molecule. In an assay that comprises the screening of such molecules, the cell that expresses one or more isoforms of Wilms' Tumor gene product is contacted with the antisense molecule suspected of inhibiting WT1 expressing cells. The ability of the antisense construct to inhibit the expression of WT1 may be assayed by methods such as measuring the levels of expression of the WT1 gene or measuring the levels of the WT1 gene product in the cell. Other suitable inhibitors include ribozymes, and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

A. In vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays.

The target may be either free in solution, fixed to a support, expressed in or on the surface of a cell. Either the target or the compound may be labeled, thereby permitting determining of binding. In another embodiment, the assay may measure the inhibition of binding of a target to a natural or artificial substrate or binding partner (such as a WT1).

Competitive binding assays can be performed in which one of the agents (WT1 for example) is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, a WT1 and washed. Bound polypeptide is detected by various methods.

Purified target, such as a WT1, can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link an active region (e.g., the C-terminus of a WT1) to a solid phase.

B. In cyto Assays

Various cell lines that express isoforms of WT1 can be utilized for screening of candidate substances. For example, cells containing a WT1 with an engineered indicator can be used to study various functional attributes of candidate compounds. In such assays, the compound would be formulated appropriately, given its biochemical nature, and contacted with a target cell.

Molecular analysis may be performed in which the function of a WT 1 and related genes may be explored. This involves assays such as those for protein expression, enzyme function, substrate utilization, mRNA expression (including differential display of whole cell or polyA RNA) and others.

### XI. QUANTITATING LEVELS OF EXPRESSION OF WT1

The levels of expression of WT1 polypeptide/protein and/or mRNA is a function of the proliferation of breast cancer cells and is thus useful for various purposes such as a prognostic method for determining the breast cancer progression, as a screening method to know whether a candidate substance is able to inhibit cancer by inhibiting the WT1 gene or gene product and also in determining the type of treatment/combination treatment that may be used on a patient depending on the efficacy of the treatment. It may also be used to determine the progress of a patient when treated with an antisense oligonucleotide therapy or to determine what type or dose of the therapeutic regimen are suitable. Therefore, some embodiments of the invention concern measuring and/or quantitation and/or estimation of levels of WT1 expression.

A. Quantitative PCR

For quantitation of a nucleic acid, reverse transcription (RT) of RNA to cDNA followed by relative quantitative or semi-quantitative PCR™ (RT-PCR™) can be used to determine the relative concentrations of specific mRNA species in a series of total cell RNAs isolated from the cancer cells.

By determining that the concentration of a specific mRNA species varies, it is shown that the gene encoding the specific mRNA species is expressed at different levels in different types of cancers.

In PCR™, the number of molecules of the amplified target DNA increase by a factor approaching two with every cycle of the reaction until some reagent becomes limiting. Thereafter, the rate of amplification becomes increasingly diminished until there is not an increase in the amplified target between cycles. If one plots a graph on which the cycle number is on the X axis and the log of the concentration of the amplified target DNA is on the Y axis, one observes that a curved line of characteristic shape is formed by connecting the plotted points.

Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ is directly proportional to the starting concentration of the target before the PCR™ was begun. By determining the concentration of the PCR™ products of the target DNA in PCR™ reactions that have completed the same number of cycles and are in their linear ranges, it is possible to determine the relative concentrations of the specific target sequence in the original DNA mixture.

If the DNA mixtures are cDNAs synthesized from RNAs isolated from different cells, the relative abundances of the specific mRNA from which the target sequence was derived can be determined for the respective tissues or cells. This direct proportionality between the concentration of the PCR™ products and the relative mRNA abundances is only true in the linear range portion of the PCR™ reaction.

The final concentration of the target DNA in the plateau portion of the curve is determined by the availability of reagents in the reaction mix and is independent the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ study to successfully determine the relative abundances of a particular mRNA species is that relative concentrations of the amplifiable cDNAs must be normalized to some independent standard. The goal of an RT-PCR™ study is to determine the abundance of a particular mRNA species relative to the average abundance of all mRNA species in the sample. In such studies, mRNAs for β-actin, asparagine synthetase and lipocortin II may be used as external and internal standards to which the relative abundance of other mRNAs are compared.

Most protocols for competitive PCR™ utilize internal PCR™ internal standards that are approximately as abundant as the target. Other studies are available that use a more conventional relative quantitative RT-PCR™ with an external standard protocol.

B. Immunodetection Methods

In still further embodiments, the present invention concerns immunodetection methods for binding, purifying, removing, quantifying or otherwise generally detecting WT1 gene product. The steps of various useful immunodetection methods have been described in the scientific literature, such as, e.g., Nakamura et al. (1987; incorporated herein by reference). Immunoassays, in their most simple and direct sense, are binding assays. Certain preferred immunoassays are the various types of enzyme linked immunosorbent assays (ELISAs), radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

1. Immunohistochemistry

Fresh-frozen and formalin-fixed, paraffin-embedded tissue blocks may be prepared from study by immunohistochemistry (IHC). For example, each tissue block consists of 50 mg of residual “pulverized” tumor. The method of preparing tissue blocks from these particulate specimens has been successfully used in previous IHC studies of various prognostic factors, e.g., in breast, and is well known to those of skill in the art (Brown et al., 1990; Abbondanzo et al., 1990; Allred et al., 1990).

Briefly, frozen-sections may be prepared by rehydrating 50 ng of frozen “pulverized” tumor at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections containing an average of about 500 remarkably intact tumor cells.

Permanent-sections may be prepared by a similar method involving rehydration of the 50 mg sample in a plastic microfuge tube; pelleting; resuspending in 10% formalin for 4 h fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

2. FACS

Fluorescent activated cell sorting, flow cytometry or flow microfluorometry provides the means of scanning individual cells for the presence of an antigen, such as WT1. The method employs instrumentation that is capable of activating, and detecting the excitation emissions of labeled cells in a liquid medium.

FACS is unique in its ability to provide a rapid, reliable, quantitative, and multiparameter analysis on either living or fixed cells. Cells would generally be obtained by biopsy, single cell suspension in blood or culture. FACS analyses would probably be most useful when desiring to analyze a number of cancer antigens at a given time, e.g., to follow an antigen profile during disease progression.

3. Western Blots

Western blotting may be used to detect inhibition of proliferation of breast cancer cell lines due to specific inhibition of WT1 protein expression. The antisense construct of the present invention may be used as high-affinity primary reagents for the identification of WT1 gene product immobilized onto a solid support matrix, such as nitrocellulose, nylon or combinations thereof. The technique of western blots is well known to a person of ordinary skill in the art.

### XII. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Materials And Methods

Cell Culture

The ERα-positive breast cancer cell lines MCF-7, BT-474, T-47D, and MDA-MB-361 (Sutherland et al., 1988; Fitzgerald et al., 1997), and the ERα-negative breast cancer cell lines SKBr-3, MDA-MB-231, MDA-MB-453, BT-20, and MDA-MB-468 (Fitzgerald et al., 1997; Love-Schimenti et al., 1996) were obtained from the American Type Culture Collection (Manassas, Va.). They were propagated in DMEM/F12 medium supplemented with 10% FCS. The human leukemia cell line K562, chosen as a positive control cell line because of its high expression of WT1 protein (Yamagami et al., 1996), was also obtained from ATCC and propagated in RPMI 1640 medium supplemented with 10% FCS. All cell lines were incubated in 95% air and 5% CO₂ at 37° C.

Western Blotting

Western blotting was used to determine the expression levels of WT1 proteins in nuclear extracts from breast cancer and leukemia cell lines since these proteins are known to localize within the nucleus (Dobashi et al., 1997). Protein concentration was determined by using the Bio-Rad DC protein concentration assay. Briefly 50 μg of proteins were subjected to electrophoresis on 12% SDS-polyacrylamide gels and transferred to nitrocellulose membranes. Immunodetection was done using rabbit antibodies specific for WT1 (C-19) from Santa Cruz Biotechnology (Santa Cruz, Calif.) and anti-rabbit secondary antibodies conjugated with horseradish peroxidase (Amersham Life Science Inc.). Protein bands were visualized by enhanced chemiluminescence (Kirkegaard & Perry Laboratories, Gaithersburg, Md.).

Preparation of Liposome—Incorporated Oligonucleotides

The following is a brief description of how oligonucleotides may be incorporated in a liposome. For details one may refer to Tari et al. (2000).

The oligonucleotides were radiolabelled with ³²p radioisotope and incorporated in DOPC lipids purchased from Avanti Polar Lipids (Alabaster, Ala.). DOPC was dissolved in tert-butanol at 20 mg/ml. Oligonucleotides are dissolved in water or DMSO at ˜8 mg/ml. Oligonucleotides are aliquoted and mixed well before adding excess tert-butanol. Because DMSO is present, tert-butanol should be added for at least 95% (v/v) so that the mixture can be well frozen in an acetone/dry ice bath before being lyophilized overnight. The lyophilized preparation is hydrated with 0.9% normal saline at a final oligonucleotide concentration of 0.1 mM.

WT1 Antisense Oligos and Cell Treatment

P-ethoxy oligos, purchased from Oligos Etc., Inc. (Wilsonville, Oreg.), were incorporated into DOPC liposomes (Tari et al., 2000). The sequence of the WT1 antisense oligos targeted against the translation initiation site is 5′-GTCGGAGCCCATTTGCTG-3′ (SEQ ID NO: 1), and the sequence of the control oligos is 5′-GGGCTTTTGAACTCTGCT-3′ (SEQ ID NO: 2) (Yamagami et al., 1996). Breast cancer and leukemia cells were plated in 96-well plates (2×10³ cells per well) in DMEM/F12 supplemented with 10% FCS and allowed to adhere overnight. Then various concentrations (0, 3, 6, or 12 μM) of liposomal WT1 antisense (L-WT1) and liposomal control (L-control) oligos were added to the cells and incubated for 72 h. Cell growth was determined by using the CellTiter 96 Aqueous nonradioactive proliferation assay (Promega, Madison, Wis.).

Light Microscopic Evaluation of Cell Growth

MCF-7 and MDA-MB-453 cells were seeded in 6-well plates (1.0×10⁵ cells per well) in DMEM/F12 medium supplemented with 10% FCS. After 24 h, the cells were treated with 12 μM L-WT1 or L-control oligos for 3 days, examined under light microscopy at 100× magnification, and photographed with Kodak gold 400 film.

RNA Purification and RT-PCR

Total RNA was prepared from the cell lines by using 1 ml of TRIzol Reagent (Life Technologies, Gaithersburg, Md.) according to the manufacturer's protocol. The pellet of RNA was dissolved in DEPC-treated-water and quantified by spectrophotometry at 260 nm. cDNA was created with Superscript II according to the manufacturer's protocol (Gibco BRL). All PCR reactions were carried out with 5 μl of cDNA, 0.2 mM dNTPs, 100 ng of each primer, 10 mM Tris-HCl (pH 8.4, 50 mM KCl, 0.01% gelatin, 1.5 mM MgCl₂), and 2.5 U of Taq DNA polymerase. PCR to detect the different WT1 isoforms was performed with primers as described by Brenner et al (Brenner et al., 1992). The thermal profile involved 35 cycles of denaturation at 94° C. for 40 s, primer annealing at 64° C. for 30 s, and extension at 72° C. for 30 s. PCR products were subjected to electrophoresis on 2% agarose gels and the reaction products were visualized with ethidium bromide and photographed under UV transillumination.

### Example 2

Expression of WT1 Protein in Breast Cancer Cell Lines

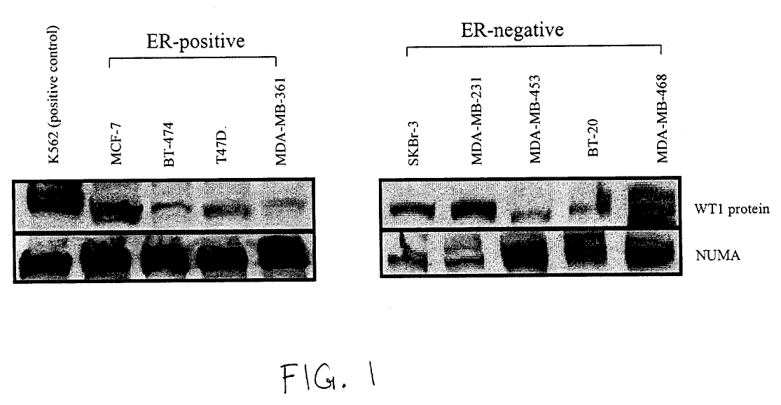

The endogenous expression of the 52-54 kDa WT1 protein in breast cancer cell lines was assessed and K562 leukemic cells were used as positive control. WT1 protein was detected in the nuclear extracts of both ER-positive and ER-negative cell lines (FIG. 1).

The results obtained indicate that WT1 protein is vital for the proliferation of breast cancer cells, regardless of whether the cells are ER-positive or ER-negative. The inventors found no correlation between the basal expression of WT1 protein and the inhibitory response to L-WT 1. Nor was any correlation evident between inhibition by L-WT1 and the status of p53 protein, as MCF-7 is the only cell line that expresses the wild-type 53 protein (Casey et al., 1991).

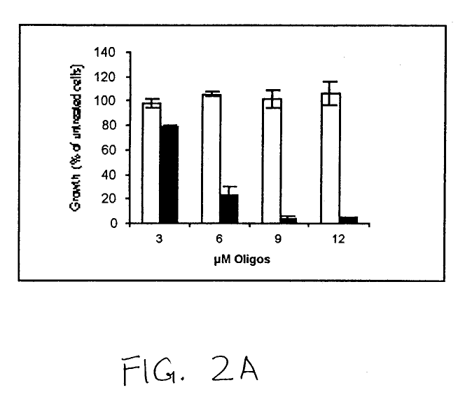

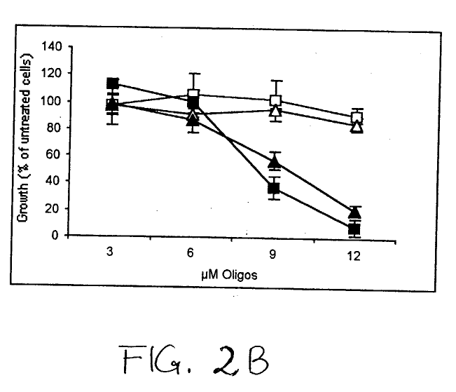

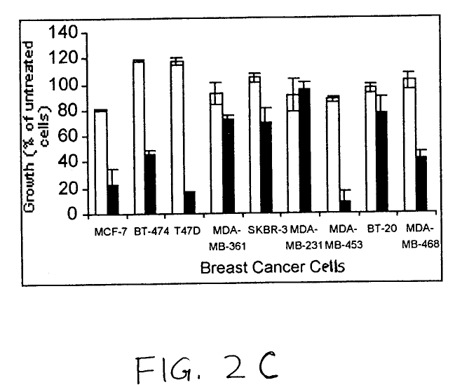

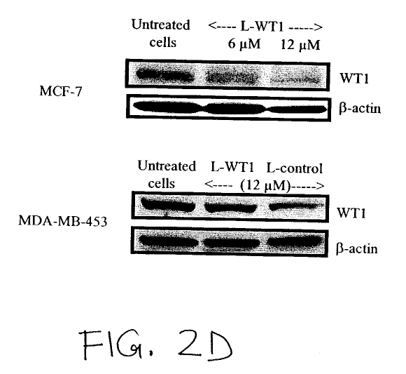

Reduction of WT1 Protein Expression Leads to Growth Inhibition of Breast Cancer. It was first verified that L-WT1 oligos could inhibit the growth of K562 leukemia cells (Yamagami et al., 1996; Algar et al., 1996) in a dose dependent manner (FIG. 2A). Next, the effect of L-WT1 on MCF-7 cells, an ER-positive cell line with high endogenous WT1 expression, and on MDA-MB-453 cells, an ER-negative cell line with low endogenous WT1 expression, was studied. L-WT1 induced dose-dependent growth inhibition in both cell lines (FIG. 2B). Maximal growth inhibition (>90%) was observed at 12 μM L-WT1; therefore, this concentration was used for the subsequent experiments. These findings were further expanded to 7 more breast cancer cell lines, the ER-positive BT-474, T-47D, and MDA-MB-361 and the ER-negative cell lines SKBr-3, MDA-MB-231, BT-20, and MDA-MB-468. L-WT1 inhibited the growth of 8 of the 9 breast cancer cell lines, with greater than 50% effects in MCF-7, T-47D, and MDA-MB-453 cells (FIG. 2C). Under the same conditions, approximately 50% growth inhibition was observed in BT-474 and MDA-MB-468 cells while less than 50% growth inhibition was observed in MDA-MB-361, SKBr-3, and BT-20 cells. No growth inhibition was observed in MDA-MB-231 cells.

Western blotting confirmed that the inhibition of proliferation in MCF-7 and MDA-MB-453 cells was due to specific inhibition of WT1 protein expression (FIG. 2D).

The inventors have detected WT1 mRNA in all cell lines and this contradicts a previous report by Loeb et al. (2001) who found WT1 mRNA in T-47D and MDA-MB-468 cells but not in MCF-7, MDA-MB-231 or SKBr-3 cells. However these investigators used one round of PCR, whereas the inventors subjected cells to reamplification by using nested PCR. The inventors' data indicated low but detectable WT1 levels in breast cancer cells, and the reamplification allowed the different WT1 isoforms to be detected as well.

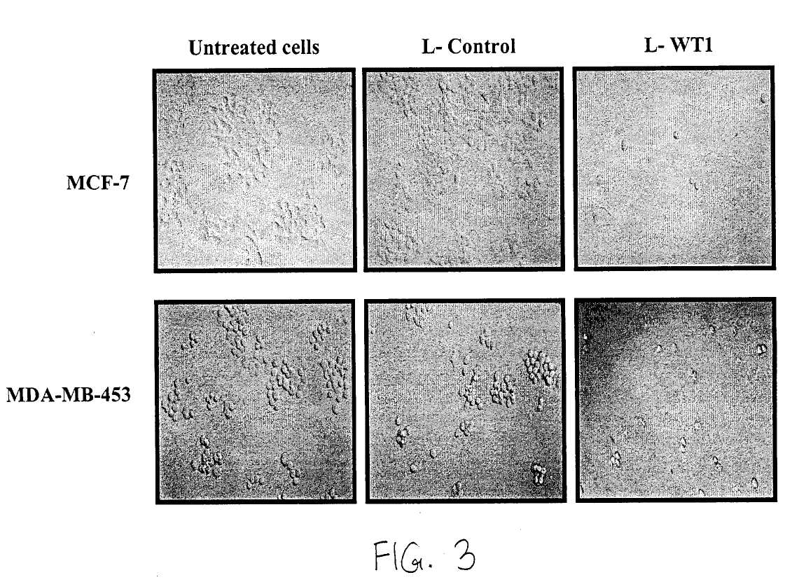

Light Microscopy. Using light microscopy, the inventors observed that L-WT1 reduced the number of MCF-7 and MDA-MB-453 cells as compared with untreated cells (FIG. 3). But L-control did not decrease the number of MCF-7 and MDA-MB-453 cells.

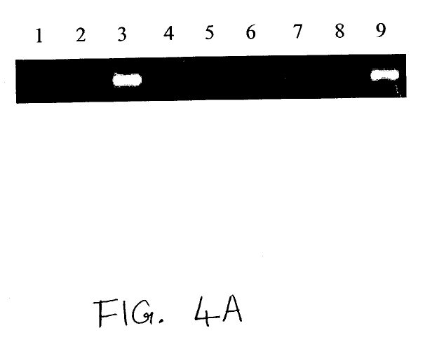

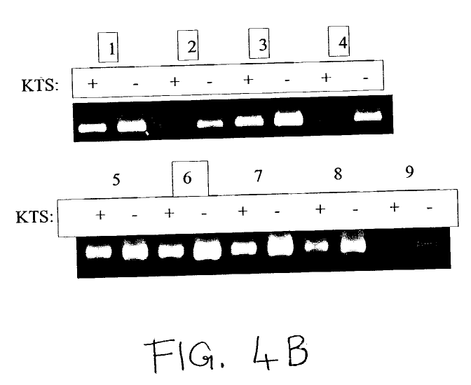

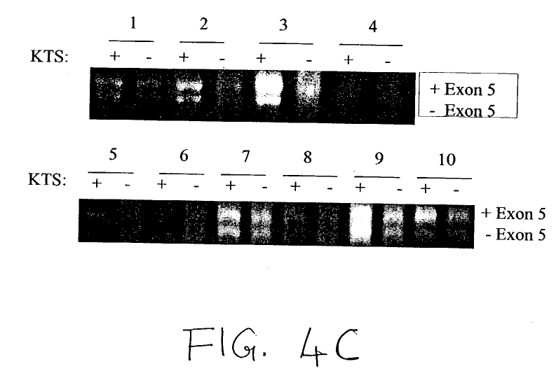

Expression of WT1 mRNA Isoforms. RT-PCR was used to determine whether expression of the total WT1 mRNA and its isoforms was associated with the growth inhibition of breast cancer cells. The highest total mRNA expression was detected in T-47D and MDA-MB-468 cells, since the PCR products of WT1 (857 bp) in these cell lines were detected in the first round of PCR (FIG. 4A). However in the other cell lines, the PCR products of WT1 were not detected until the second round of PCR.

To identify the various WT1 mRNA isoforms, the inevntors first amplified the KTS region with specific primers to exon 7 and primers to the KTS− or KTS+ areas in exon 9. All cell lines produce two products, but the KTS− isoform was more abundant than the KTS+ isoform (FIG. 4B). To detect exon 5 isoforms, primers to exon 1 and primers to KTS− or KTS+ isoforms were used (FIG. 4C), all four WT1 isoforms were detected in the control K562 cells and in the ER-positive cells. Among the four ER-positive cells, MDA-MB-361 cells had the lowest expression of these isoforms. All four isoforms were also detected in two of the five ER-negative cell lines MDA-MB-453 and MDA-MB-468. However, only the exon 5+/KTS+ and exon 5−/KTS− isoforms were detected in the SKBr-3 cells, and only the exon 5+/KTS+ and exon 5+/KTS− isoforms were detected in the BT-20 cells. No PCR products were observed in the MDA-MB-231 cells.

As described earlier in the description, the WT1 splicing isoforms have different biological activities (Lee et al., 2001). The KTS− isoforms have transactivational properties in some genes that are involved in cell growth and differentiation, whereas the KTS+ isoforms have a potential role in RNA processing (Lee et al., 2001). Exon 5 may function as a repressor domain or as an activator domain, depending on which proteins are interacting with WT1 (Richard et al., 2001). In the inventors' study, all five cell lines in which L-WT1 led to ≧50% growth inhibition contain all four WT1 isoforms. But the two cell lines that were little affected by L-WT1 expressed only two WT1 isoforms, and the one cell line that was not affected by L-WT1 expressed no WT1 isoforms. These data show that the regulation of breast cancer cell growth by WT1 protein may depend on the expression of all four WT1 isoforms.

All of the methods and compositions disclosed and claimed herein can be made and executed without undue experimentation in light of the present disclosure. While the compositions and methods of this invention have been described in terms of preferred embodiments, it will be apparent to those of skill in the art that variations may be applied to the methods and compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

U.S. Pat. No. 4,659,774

U.S. Pat. No. 4,682,195

U.S. Pat. No. 4,682,195

U.S. Pat. No. 4,683,202

U.S. Pat. No. 4,816,571

U.S. Pat. No. 4,959,463

U.S. Pat. No. 5,141,813

U.S. Pat. No. 5,264,566

U.S. Pat. No. 5,428,148

U.S. Pat. No. 5,554,744

U.S. Pat. No. 5,574,146

U.S. Pat. No. 5,602,244

U.S. Pat. No. 5,645,897

U.S. Pat. No. 5,705,629

U.S. Pat. No. 6,034,235

Abbondanzo, “Paraffin immunohistochemistry as an adjunct to hematopathology,” Ann Diagn Pathol, 3(5):318-327, 1999.

Algar, Khromykh, Smith, Blackburn, Bryson, Smith, “A WT1 antisense oligonucleotide inhibits proliferation and induces apoptosis in myeloid leukaemia cell lines,” Oncogene, 12:1005-1014, 1996.

Allred, Bustamante, Daniel, Gaskill, Cruz Jr., “Immunocytochemical analysis of estrogen receptors in human breast carcinomas. Evaluation of 130 cases and review of the literature regarding concordance with biochemical assay and clinical relevance,” Arch Surg, 125(1):107-13, 1990.

Angel, Bauman, Stein, Dellus, Rahmsdorf, and Herrlich, “12-0-tetradecanoyl-phorbol-13-acetate induction of the human collagenase gene is mediated by an inducible enhancer element located in the 5′ flanking region,” Mol. Cell. Biol., 7:2256, 1987.

Angel, Imagawa, Chiu, Stein, Imbra, Rahmsdorf, Jonat, Herrlich, and Karin, “Phorbol ester-inducible genes contain a common cis element recognized by a TPA-modulated trans-acting factor,” Cell, 49:729, 1987

Atchison and Perry, “Tandem ε immunoglobulin promoters are equally active in the presence of the ε enhancer: Implications for model of enhancer function,” Cell, 46:253, 1986.

Atchison and Perry, “The role of the K enhancer and its binding factor nf-ε β in the developmental regulation of ε gene transcription,” Cell, 48:121, 1987.

Baichwal and Sugden, “Vectors for gene transfer derived from animal DNA viruses: Transient and stable expression of transferred genes,” In: Kucherlapati R, (ed.) Gene transfer. New York: Plenum Press, pp. 117-148, 1986.

Bakhshi, Jensen, Goldman, Wright, McBride, Epstein, Korsmeyer, “Cloning the chromosomal breakpoint of t(14;18) human lymphomas: clustering around JH on chromosome 14 and near a transcriptional unit on 18,” Cell, 41(3):899-906, 1985.

Banerji, Olson, Schaffner, “A lymphocyte-specific cellular enhancer is located downstream of the joining region in immunoglobulin heavy chain genes,” Cell, 33(3):729-740, 1983.

Banerji, Rusconi, Schaffner, “Expression of a β-globin gene is enhanced by remote SV40 DNA sequences,” Cell, 27(2 Pt 1):299-308, 1981.

Bangham, Standish, Watkins, “Diffusion of univalent ions across the lamellae of swollen phospholipids,“J. Mol. Biol., 13(1):238-52, 1965.

Benvenisty and Neshif, “Direct introduction of genes into rats and expression of the genes,” Proc. Natl. Acad. Sci. USA, 83(24):9551-9555, 1986.

Berkhout, Silverman, Jeang, “Tat trans-activates the human immunodeficiency virus through a nascent RNA target,” Cell, 59:273-282, 1989.

Blanar, Baldwin, Flavell, and Sharp, “A gamma-interferon-induced factor that binds the interferon response sequence of the MHC class I gene, H-2Kb,” EMBO J., 8:1139, 1989.

Bodine and Ley, “An enhancer element lies 3′ to the human a gamma globin gene,” EMBO J, 6:2997, 1987.

Boshart, Weber, Jahn, Dorsch-Hasler, Fleckenstein, and Schaffner, “A very strong enhancer is located upstream of an immediate early gene of human cytomegalovirus,” Cell, 41:521, 1985.

Bosze, Thiesen, Charnay, “A transcriptional enhancer with specificity for erythroid cells is located in the long terminal repeat of the Friend murine leukemia virus,” EMBO J., 5(7):1615-1623, 1986.

Braddock, Chambers, Wilson, Esnouf, Adams, Kingsman, and Kingsman, “HIV-I Tat activates presynthesized RNA in the nucleus,” Cell, 58:269, 1989.

Breitbart, Andreadis, Nadal-Ginard, Annu. Rev. Biochem., 56:467-495, 1987.

Brenner, Wildhardt, Schneider, Royer-Pokora, “RNA polymerase chain reaction detects different levels of four alternatively spliced WT1 transcripts in Wilms' tumors,” Oncogene, 7:1431-1433, 1992.

Brown, Linsley, Horn, “Development and evaluation of monoclonal antibody-based immunoassays: breast carcinoma-associated mucins as tumor markers,” Immunol Ser, 53:69-82, 1990.

Bulla and Siddiqui, “The hepatitis B virus enhancer modulates transcription of the hepatitis B virus surface-antigen gene from an internal location,” J. Virol., 62:1437, 1986.

Caldas, Hahn, da Costa, Redston, Schutte, Seymour, Weinstein, Hruban, Yeo, Kern, “Frequent somatic mutations and homozygous deletions of the p16 (MTS1) gene in pancreatic adenocarcinoma,” Nat. Genet., 8(1):27-32, 1994.

Call, Glaser, Ito, Buckler, Pelletier, Haver, Rose, Kral, Yeger, Lewis, Jones, Housman, “Isolation and charcterization of a zinc finger polypetide gene at the human chromosome 11 Wilms' tumor locus,” Cell, 60:509-520, 1990.

Campbell and Villarreal, “Functional analysis of the individual enhancer core sequences of polyoma virus: cell-specific uncoupling of DNA replication from transcription,” Mol. Cell. Biol., 8:1993, 1988.

Campere and Tilghman, “Postnatal repression of the α-fetoprotein gene is enhancer independent,” Genes and Dev., 3:537, 1989.

Campo, Spandidos, Lang, Wilkie, “Transcriptional control signals in the genome of bovine papilloma virus type 1,” Nature, 303:77, 1983.

Casey, Lo-Hsueh, Lopez, Vogelstein, Stanbridge, “Growth suppression of human breast cancer cells by the introduction of a wild-type p53 gene,” Oncogene, 6:1791-1797, 1991.

Celander and Haseltine, “Glucocorticoid regulation of murine leukemia virus transcription elements is specified by determinants within the viral enhancer region,” J. Virology, 61:269, 1987.

Celander, Hsu, and Haseltine, “Regulatory Elements Within the Murine Leukemia Virus Enhancer Regions Mediate Glucocorticoid Responsiveness,” J. Virology, 62:1314, 1988.

Chandler, Maler, and Yamamoto, “DNA sequences bound specifically by glucocorticoid receptor in vitro render a heterlogous promoter hormone responsive in vivo,” Cell, 33:489, 1983.

Chang et al., “Foreign gene delivery and expression in hepatocytes using a hepatitis B virus vector,” Hepatology, 14:134A, 1991.

Chang, Erwin, and Lee, “Glucose-regulated Protein (GRP94 and GRP78) Genes Share Common Regulatory Domains and are Coordinately Regulated by Common Trans-acting Factors,” Mol. Cell. Biol., 9:2153, 1989.

Chatterjee, Lee, Rentoumis, and Jameson, “Negative regulation of the thyroid-stimulating hormone alpha gene by thyroid hormone: receptor interaction adjacent to the TATA box,” Proc Natl. Acad Sci. U.S.A., 86:9114, 1989.

Chen and Okayama, “High-efficiency transformation of mammalian cells by plasmid DNA,” Mol. Cell Biol., 7(8):2745-2752, 1987.

Cheng, Jhanwar, Klein, Bell, Lee, Altomare, Nobori, Olopade, Buckler, Testa Jr., “p16 alterations, and deletion mapping of 9p21-p22 in malignant mesothelioma,” Cancer Res., 54(21):5547-5551, 1994.

Choi, Chen, Kriegler, and Roninson, “An altered pattern of cross-resistance in multi-drug-resistant human cells results from spontaneous mutations in the mdr-1 (p-glycoprotein) gene,” Cell, 53:519, 1988.

Cleary and Sklar, “Nucleotide sequence of a t(14;18) chromosomal breakpoint in follicular lymphoma and demonstration of a breakpoint-cluster region near a transcriptionally active locus on chromosome 18,” Proc. Natl. Acad. Sci. USA, (21):7439-7443, 1985.

Cleary, Galili, Sklar, “Detection of a second t(14;18) breakpoint cluster region in human follicular lymphomas,” J. Exp. Med., 164(1):315-320, 1986.

Coffin, “Retroviridae and their replication,” In: Virology, Fields et al., eds., Raven Press, New York, pp. 1437-1500,1990.

Cohen, Walter, and Levinson, “A Repetitive Sequence Element 3′ of the Human c-Ha-ras1 Gene Has Enhancer Activity,” J. Cell. Physiol., 5:75, 1987.

Costa, Lai, Grayson, and Darnell, “The cell-specific enhancer of the mouse transthyretin (prealbumin) gene binds a common factor at one site and a liver-specific factor(s) at two other sites,” Mol. Cell. Biol., 8:81-90, 1988.

Coupar, Andrew, Boyle, “A general method for the construction of recombinant vaccinia viruses expressing multiple foreign genes,” Gene, 68:1-10, 1988.

Cripe, Haugen, Turk, Tabatabai, Schmid, Durst, Gissmann, Roman, and Turek, “Transcriptional Regulation of the Human Papilloma Virus-16 E6-E7 Promoter by a Keratinocyte-Dependent Enhancer, and by Viral E2 Trans-Activator and Repressor Gene Products: Implications for Cervical Carcinogenesis,” EMBO J., 6:3745, 1987.

Culotta and Hamer, “Fine Mapping of a Mouse Met allothionein Gene Met al-Response Element,” Mol. Cell. Biol., 9:1376-1380, 1989.

Dandolo, Blangy, and Kamen, “Regulation of Polyma Virus Transcription in Murine Embryonal Carcinoma Cells,” J. Virology, 47:55-64, 1983.

De Villiers, Schaffnuer, Tyndall, Lupton, and Kamen, “Polyoma Virus DNA Replication Requires an Enhancer,” Nature, 312(5991):242-246, 1984.

Deamer and Uster, “Liposome Preparation: Methods and Mechanisms,” LIPOSOMES, M. Ostro ed. 1983.

Deschamps, Meijlink, and Verma, “Identification of a transcriptional enhancer element upstream from the proto-oncogene fos,” Science, 230:1174-1177, 1985.

Dobashi, Kudoh, Ishidate, Shoji, Toyoshima, Akiyama, “The Wilms tumor protein is persistently associated with the nuclear matrix throughout the cell cycle,” Mol. Cell Biochem., 171:121-126, 1997.

Dubensky, Campbell, Villarreal, “Direct transfection of viral and plasmid DNA into the liver or spleen of mice,” Proc. Nat'l Acad. Sci. USA, 81:7529-7533, 1984.

Edbrooke, Burt, Cheshire, and Woo, “Identification of cis-acting sequences responsible for phorbol ester induction of human serum amyloid a gene expression via a nuclear-factor-kappa β-like transcription factor,” Mol. Cell. Biol., 9:1908-1916, 1989.

Edlund, Walker, Barr, and Rutter, “Cell-specific expression of the rat insulin gene: evidence for role of two distinct 5′ flanking elements,” Science, 230:912-916, 1985.

European Pat. App. No. 266,032

Fecheimer et al., “Transfection of mammalian cells with plasmid DNA by scrape loading and sonication loading,” Proc. Nat'l Acad. Sci. USA, 76:3348-52, 1979.

Feng and Holland, “HIV-I Tat Trans-Activation Requires the Loop Sequence Within Tar,” Nature, 334:6178, 1988.

Firak and Subramanian, “Minimal Transcription Enhancer of Simian Virus 40 is a 74-Base-Pair Sequence that Has Interacting Domains,” Mol. Cell. Biol., 6:3667, 1986.

Fitzgerald, Teng, Chandraratna, Heyman, Allegretto, “Retinoic acid receptor a expression correlates with retinoid-induced growth inhibition of human breast cancer cells regardless of estrogen receptor status,” Cancer Res., 57:2642-2650, 1997.

Foecking and Hofstetter, “Powerful and Versatile Enhancer-Promoter Unit for Mammalian Expression Vectors,” Gene, 45(1):101-105, 1986.

Friedman et al., “CCAAT/enhancer-binding protein activates the promoter of the serum albumin gene in cultured hepatoma cells,” Genes Devel. 3:1314, 1989.

Froehler, Ng, Matteucci, “Synthesis of DNA via deoxynucleoside H-phosphonate intermediates,” Nucleic Acids Res., 14(13):5399-5407, 1986.

Fujita, Shibuya, Hotta, Yamanishi, and Taniguchi, “Interferon-Beta Gene Regulation: Tandemly Repeated Sequences of a Synthetic 6-bp Oligomer Function as a Virus-Inducible Enhancer,” Cell, 49:357, 1987.

Gessler, Poustka, Cavenee, Neve, Orkin, Bruns, “Homozygous deletion in Wilms tumours of a zinc-finger gene identified by chromosome jumping,” Nature, 343:774-778, 1990.

Ghosh and Bachhawat, Targeting of Liposomes to Hepatocytes. In: Liver Diseases, Targeted Diagnosis and Therapy Using Specific Receptors and Ligands. Wu et al., eds., Marcel Dekker, New York, pp. 87-104, 1991.

Gilles, Morris, Oi, and Tonegawa, “A tissue-specific transcription enhancer element is located in the major intron of a rearranged immunoglobulin heavy-chain gene,” Cell, 33:717, 1983.

Gloss, Bernard, Seedorf, and Klock, “The upstream regulatory region of the human papilloma virus-16 contains an E2 protein-independent enhancer which is specific for cervical carcinoma cells and regulated by glucocorticoid hormones,” EMBO J., 6:3735, 1987.

Godbout, Ingram, and Tilghman, “Fine-Structure Mapping of the Three Mouse Alpha-Fetoprotein Gene Enhancers,” Mol. Cell. Biol., 8:1169, 1988.

Goodbourn and Maniatis, “Overlapping Positive and Negative Regulatory Domains of the Human β-Interferon Gene,” Proc. Natl. Acad. Sci. USA, 85:1447, 1988.

Goodbourn, Burstein, and Maniatis, “The Human Beta-Interferon Gene Enhancer is Under Negative Control,” Cell, 45:601, 1986.

Gopal, “Gene transfer method for transient gene expression, stable transformation, and cotransformation of suspension cell cultures,” Mol. Cell Biol., 5:1188-1190, 1985.

Gossen and Bujard, Proc. Nat'l Acad. Sci. USA, 89:5547-5551, 1992.

Gossen et al., Science, 268:1766-1769, 1995.

Graham and van der Eb, “A new technique for the assay of infectivity of human adenovirus 5 DNA”, Virology, 52:456-467, 1973.

Graham et al., “Characteristics of a human cell line transformed by DNA from human adenovirus type 5 DNA”, J. Gen. Virol., 36:59-72, 1977.

Greene, Bohnlein, and Ballard, “HIV-1, and Normal T-Cell Growth: Transcriptional Strategies and Surprises,” Immunology Today, 10:272, 1989

Gregoriadis, In: Drug Carriers In Biology And Medicine, Gregoriadis (ed.), 287-341, 1979.

Grosschedl and Baltimore, “Cell-type specificity of immunoglobulin gene expression is regulated by at least three dna sequence elements,” Cell, 41:885, 1985.

Grunhaus, Cho, Horwitz, “Association of vaccinia virus-expressed adenovirus E3-19K glycoprotein with class I MHC and its effects on virulence in a murine pneumonia model,” Seminar in Virology, 200(2):535-546, 1992.

Guan, Liu, Xu, Wang, “A point mutation within exon 5 of the WT1 gene of a sporadic unilateral Wilms' tumor alters gene function,” Cancer Res., 58:4180-4184, 1998.

Haber, Buckler, Glaser, Call, Pelletier, Sohn, Douglass, Housman, “An internal deletion within an 11p13 zinc finger gene contributes to the development of Wilms' tumor,” Cell, 61:1257-1269, 1990.

Haber, Sohn, Buckler, Pelletier, Call, Housman, “Alternative splicing and genomic structure of the Wilms tumor gene WT1,” Proc. Natl. Acad. Sci. USA, 88:9618-9622, 1991.

Harland and Weintraub, “Translation of mRNA injected into Xenopus oocytes is specifically inhibited by antisense RNA,” J. Cell Biol., 101:1094-1099, 1985.

Haslinger and Karin, “Upstream Promoter Element of the Human Met allothionein-II Gene Can Act Like an Enhancer Element,” Proc Natl. Acad. Sci. U.S.A., 82:8572, 1985.

Hauber and Cullen, “Mutational Analysis of the Trans-Activiation-Responsive Region of the Human Immunodeficiency Virus Type I Long Terminal Repeat,” J. Virology, 62:673, 1988.

Hen, Borrelli, Fromental, Sassone-Corsi, and Chambon, “A Mutated Polyoma Virus Enhancer Which is Active in Undifferentiated Embryonal Carcinoma Cells is not Repressed by Adenovirus-2 E1A Products,” Nature, 321:249, 1986.

Hensel, Meichle, Pfizenmaier, and Kronke, “PMA-responsive 5′ flanking sequences of the human TNF gene,” Lymphokine Res., 8:347, 1989.

Hermonat and Muzycska, “Use of adeno-associated virus as a mammalian DNA cloning vector: transduction of neomycin resistance into mammalian tissue culture cells,” Proc. Nat'l Acad. Sci. USA, 81:6466-6470, 1984.

Herr and Clarke, “The SV40 Enhancer is Composed of Multiple Functional Elements That Can Compensate for One Another,” Cell, 45:461, 1986.

Hirochika, Browker, and Chow, “Enhancers and Trans-Acting E2 Transcriptional Factors of Papilloma Viruses,” J. Virol., 61:2599, 1987.

Hirsch, Gaugler, Deagostini-Bauzin, Bally-Cuilf, and Gordis, “Identification of positive and negative regulatory elements governing cell-type-specific expression of the neural-cell-adhesion-molecule gene,” Mol. Cell. Biol., 10:1959, 1990.

Holbrook, Gulino, and Ruscetti, “cis-Acting Transcriptional Regulatory Sequences in the Gibbon Ape Leukemia Virus (GALV) Long Terminal Repeat,” Virology, 157:211, 1987.

Hollstein, Sidransky, Vogelstein, Harris, “p53 mutations in human cancers,” Science, 253(5015):49-53, 1991.

Horlick and Benfield, “The Upstream Muscle-Specific Enhancer of the Rat Muscle Creatine Kinase Gene is Composed of Multiple Elements,” Mol. Cell. Biol., 9:2396, 1989.

Horwich, Furtak, Pugh, Summers, “Synthesis of hepadnavirus particles that contain replication-defective duck hepatitis B virus genomes in cultured HuH7 cells,” Virol., 64:642-650, 1990.

Huang, Ostrowski, Berard, and Hagar, “Glucocorticoid regulation of the ha-musv p21 gene conferred by sequences from mouse mammary tumor virus,” Cell, 27:245, 1981.

Hug, Costas, Staeheli, Aebi, and Weissmann, “Organization of the Murine Mx Gene and Characterization of its Interferon- and Virus-Inducible Promoter,” Mol. Cell. Biol., 8:3065, 1988.

Hussussian, Struewing, Goldstein, Higgins, Ally, Sheahan, Clark Jr, Tucker, Dracopoli, “Germline p16 mutations in familial melanoma,” Nat. Genet., 8(1):15-21, 1994.

Hwang, Lim, and Chae, “Characterization of the S-Phase-Specific Transcription Regulatory Elements in a DNA-Replication-Independent Testis-Specific H2B (TH2B) Histone Gene,” Mol. Cell. Biol., 10:585, 1990.

Imagawa, Chiu, and Karin, “Transcription Factor AP-2 Mediates Induction by Two Different Signal-Transduction Pathways: Protein Kinase C. and cAMP,” Cell, 51:251, 1987.

Imbra and Karin, “Phorbol Ester Induces the Transcriptional Stimulatory Activity of the SV40 Enhancer,” Nature, 323:555, 1986.

Imler, Lemaire, Wasvlyk, and Waslyk, “Negative Regulation Contributes to Tissue Specificity of the Immunoglobulin Heavy-Chain Enhancer,” Mol. Cell. Biol, 7:2558, 1987.

Imperiale and Nevins, “Adenovirus 5 E2 Transcription Unit: an ElA-Inducible Promoter with an Essential Element that Functions Independently of Position or Orientation,” Mol. Cell. Biol., 4:875, 1984.

Inoue, Sugiyama, Ogawa, Nakagawa, Yamagami, Miwa, Kita, Hiraoka, Masaoka, Nasu, Kyo, Dohy, Nakauchi, Ishidate, Akiyama, Kishimoto, “WT1 as a new prognostic factor and a new marker for the detection of minimal residual disease in acute leukemia,” Blood, 84:3071-3079, 1994.

Jakobovits, Smith, Jakobovits, and Capon, “A Discrete Element 3′ of Human Immunodeficiency Virus 1 (HIV-1) and HIV-2 mRNA Initiation Sites Mediates Transcriptional Activation by an HIV Trans-Activator,” Mol. Cell. Biol., 8:2555, 1988.

Jameel and Siddiqui, “The Human Hepatitis B Virus Enhancer Requires Transacting Cellular Factor(s) for Activity,” Mol. Cell. Biol., 6:710, 1986.

Jaynes, Johnson, Buskin, Gartside, and Hauschka, “The Muscle Creatine Kinase Gene is Regulated by Multiple Upstream Elements, Including a Muscle-Specific Enhancer,” Mol. Cell. Biol., 8:62, 1988.

Johnson, Wold, and Hauschka, “Muscle creatine kinase sequence elements regulating skeletal and cardiac muscle expression in transgenic mice,” Mol. Cell. Biol., 9:3393, 1989.

Joseph, LeBeau, Jamieson, Acharya, Shows, Rowley, Sukhatme, Proc. Natl. Acad. Sci. USA, 85:7164-7168, 1988.

Kadesch and Berg, “Effects of the Position of the Simian Virus 40 Enhancer on Expression of Multiple Transcription Units in a Single Plasmid,” Mol. Cell. Biol., 6:2593, 1986.

Kamb, Shattuck-Eidens, Eeles, Liu, Gruis, Ding, Hussey, Tran, Miki, Weaver-Feldhaus et al., “Analysis of the p16 gene (CDKN2) as a candidate for the chromosome 9p melanoma susceptibility locus,” Nat. Genet., 8(1):23-2, 1994.

Kaneda, Iwai, Uchida, “Increased expression of DNA cointroduced with nuclear protein in adult rat liver,” Science, 243:375-378, 1989.

Karin, Haslinger, Heguy, Dietlin, and Cooke, “Met al-Responsive Elements Act as Positive Modulators of Human Met allothionein-IIA Enhancer Activity,” Mol. Cell. Biol., 7:606, 1987.

Katinka, Vasseur, Montreau, Yaniv, and Blangy, “Polyoma DNA Sequences Involved in the Control of Viral Gene Expression in Murine Embryonal Carcinoma Cells,” Nature, 290:720, 1981.

Katinka, Yaniv, Vasseur, and Blangy, “Expression of Polyoma Early Functions in Mouse Embryonal Carcinoma Cells Depends on Sequence Rearrangements in the Beginning of the Late Region,” Cell, 20:393, 1980.

Kato, Nakanishi, Kaneda, Uchida, Okada, “Expression of hepatitis B virus surface antigen in adult rat liver. Co-introduction of DNA and nuclear protein by a simplified liposome method,” J. Biol. Chem., 266:3361-3364, 1991.

Kawamoto, Makino, Niw, Sugiyama, Kimura, Anemura, Nakata, and Kakunaga, “Identification of the Human Beta-Actin Enhancer and its Binding Factor,” Mol. Cell. Biol., 8:267, 1988.

Kerr, Wyllie, Currie, “Apoptosis: a basic biological phenomenon with wide-ranging implications in tissue kinetics,” Br. J. Cancer, 26(4):239-257, 1972.

Kiledjian, Su, Kadesch, “Identification and characterization of two functional domains within the murine heavy-chain enhancer,” Mol. Cell. Biol., 8:145, 1988.

Klamt, Koziell, Poulat, Wieacker, Scambler, Berta, Gessler, “Frasier syndrome is caused by defective alternative splicing of WT1 leading to an altered ratio of WT1±KTS splice isoforms,” Hum. Mol. Genet., 7:709-714, 1998.

Klamut, Gangopadyhay, Worton, and Ray, “Molecular and Functional Analysis of the Muscle-Specific Promoter Region of the Duchenne Muscular Dystrophy Gene,” Mol. Cell. Biol., 10:193, 1990.

Klein et al., “High-velocity microprojectiles for delivering nucleic acids into living cells,” Nature, 327:70-73, 1987.

Koch, Benoist, and Mathis, “Anatomy of a new β-cell-specific enhancer,” Mol. Cell. Biol., 9:303, 1989.

Kriegler and Botchan, “A retrovirus LTR contains a new type of eukaryotic regulatory element,” In: Eukaryotic Viral Vectors, Gluzman (ed.), Cold Spring Harbor, Cold Spring Harbor Laboratory, NY, 1982.

Kriegler and Botchan, “Enhanced transformation by a simian virus 40 recombinant virus containing a Harvey murine sarcoma virus long terminal repeat,” Mol. Cell. Biol. 3:325, 1983.

Kriegler et al., “Promoter substitution and enhancer augmentation increases the penetrance of the sv40 a gene to levels comparable to that of the harvey murine sarcoma virus ras gene in morphologic transformation,” In: Gene Expression, Alan Liss (Ed.), Hamer and Rosenberg, New York, 1983.

Kriegler et al., “Viral Integration and Early Gene Expression Both Affect the Efficiency of SV40 Transformation of Murine Cells: Biochemical and Biological Characterization of an SV40 Retrovirus,” In: Cancer Cells 2/Oncogenes and Viral Genes, Van de Woude et al. eds, Cold Spring Harbor: Cold Spring Harbor Laboratory, 1984.

Kriegler, Perez, Hardy and Botchan, “Transformation mediated by the sv40 t antigens: separation of the overlapping sv40 early genes with a retroviral vector,” Cell, 38:483, 1984.

Kuhl, De La Fuenta, Chaturvedi, Parinool, Ryals, Meyer, and Weissman, “Reversible Silencing of Enhancers by Sequences Derived From the Human IFN-alpha Promoter,” Cell, 50:1057, 1987.

Kunz, Zimmerman, Heisig, and Heinrich, “Identification of the Promoter Sequences Involved in the Interleukin-6-Dependent Expression of the Rat Alpha-2-Macroglobulin Gene,” Nucl. Acids Res., 17:1121, 1989.

Larsen, Hamey, and Moore, “Repression medaites cell-type-specific expression of the rat growth hormone gene,” Proc Natl. Acad. Sci. USA., 83:8283, 1986.

Laspia, Rice, and Mathews, “HIV-1 Tat protein increases transcriptional initiation and stabilizes elongation,” Cell, 59:283, 1989.

Latimer, Berger, and Baumann, “Highly conserved upstream regions of the alpha..sub.1-antitrypsin gene in two mouse species govern liver-specific expression by different mechanisms,” Mol. Cell. Biol., 10:760, 1990.

Lee and Haber, “Wilms Tumor and the WT1 gene,” Exp Cell Res., 264:74-99, 2001.

Lee, Mulligan, Berg, and Ringold, “Glucocorticoids Regulate Expression of Dihydrofolate Reductase cDNA in Mouse Mammary Tumor Virus Chimaeric Plasmids,” Nature, 294:228, 1981.

Levinson, Khoury, VanDeWoude, and Gruss, “Activation of SV40 Genome by 72-Base-Pair Tandem Repeats of Moloney Sarcoma Virus,” Nature, 295:79, 1982.

Lin, Cross, Halden, Dragos, Toledano, and Leonard, “Delineation of an enhancerlike positive regulatory element in the interleukin-2 receptor .alpha.-chain gene,” Mol. Cell. Biol., 10:850, 1990.

Little, Prosser, Condie, Smiith, Van Heyningen, Hastie, “Zine finger point mutations within the WT1 gene in Wilms tumor patients,” Proc. Natl. Acad. Sci, USA, 89:4791-4795, 1992.

Liu, Wang, Deuel, Xu, “Imbalanced expression of functionally different WT1 isoforms may contribute to sporadic unilateral Wilms' tumor,. Biochem. Biophys. Res. Commun., 254:197-199, 1999.

Loeb, Evron, Patel, Sharma, Niranjan, Buluwela, Weitzman, Korz, Sukumar, Wilms' tumor suppresor gene (WT1) is expressed in primary breast tumors despite tumor-specific promoter methylation,” Cancer Res., 61:921-925, 2001.

Love-Schimenti, Gibson, Ratnam, Bikle, “Antiestrogen potentiation of antiproliferative effects of vitamin D3 analogues in breast cancer cells,” Cancer Res., 56:2789-2794, 1996.

Luria, Gross, Horowitz, and Givol, “Promoter Enhancer Elements in the Rearranged Alpha-Chain Gene of the Human T-Cell Receptor,” EMBO J., 6:3307, 1987.

Lusky and Botchan, “Transient Replication of Bovine Papilloma Virus Type 1 Plasmids: cis and trans Requirements,” Proc Natl. Acad. Sci. U.S.A., 83:3609, 1986.

Lusky, Berg, Weiher, and Botchan, “Bovine Papilloma Virus Contains an Activator of Gene Expression at the Distal End of the Early Transcription Unit,” Mol. Cell. Biol. 3:1108, 1983.

Majors and Varmus, “A small region of the mouse mammary tumor virus long terminal repeat confers glucocorticoid hormone regulation on a linked heterologous gene,” Proc. Natl. Acad. Sci. USA, 80:5866, 1983.

Mann, Mulligan, Baltimore, “Construction of a retrovirus packaging mutant and its use to produce helper-free defective retrovirus,” Cell, 33:153-159, 1983.

Marsters et al., Recent Prog Horm Res, 54:225-234, 1999.

McNeall, Sanchez, Gray, Chesterman, and Sleigh, “Hyperinducible Gene Expression From a Met allotionein Promoter Containing Additional Metal-Responsive Elements,” Gene, 76:81, 1989.

Miksicek, Heber, Schmid, Danesch, Posseckert, Beato, and Schutz, “Glucocorticoid Responsiveness of the Transcriptional Enhancer of Moloney Murine Sarcoma Virus,” Cell, 46:203, 1986.

Mitchell, et al., Ann NY Acad Sci., 690:153-66, 1993.

Mitchell, et al., J. Clin Oncol., 8(5):856-69, 1990.

Mordacq and Linzer, “Co-localization of elements required for phorbol ester stimulation and glucocorticoid repression of proliferin gene expression,” Genes and Dev., 3:760, 1989.

Moreau, Hen, Wasylyk, Everett, Gaub, and Chambon, “The SV40 base-repair repeat has a striking effect on gene expression both in sv40 and other chimeric recombinants,” Nucl. Acids Res., 9:6047, 1981.

Morton and Ravindranath, Ann Surg., 216(4):463-82, 1992.

Musesing, Smith, and Capon, “Regulation of mRNA Accumulation by a Human Immunodeficiency Virus Trans-Activator Protein,” Cell, 48:691, 1987.

Nakamura et al., In: Handbook of Experimental Immunology (4^{th} Ed.), Weir, Herzenberg, Blackwell, Herzenberg, (eds). Vol. 1, Chapter 27, Blackwell Scientific Publ., Oxford, 1987.

Ng, Gunning, Liu, Leavitt, and Kedes, “Regulation of the Human Beta-Actin Promoter by Upstream and Intron Domains,” Nuc. Acids Res., 17:601, 1989.

Nicolas and Rubinstein, In: Vectors: A survey of molecular cloning vectors and their uses, Stoneham: Butterworth, pp. 494-513, 1988.

Nicolau, Legrand, Grosse, “Liposomes as carriers for in vivo gene transfer and expression,” Methods Enzymol., 149:157-176, 1987.

Nobori, Miura, Wu, Lois, Takabayashi, Carson, “Deletions of the cyclin-dependent kinase-4 inhibitor gene in multiple human cancers,” Nature, 368(6473):753-756, 1994.

Oji Y, et al., “Expression of the Wilms' tumor gene WT1 in solid tumors and its involvement in tumor cell growth,” Jpn J Cancer Res, February;90(2):194-204, 1999.

Okamoto, Demetrick, Spillare, Hagiwara, Hussain, Bennett, Forrester, Gerwin, Serrano, Beach et al., “Mutations and altered expression of p16INK4 in human cancer,” Proc Natl Acad Sci USA, 1(23):11045-11049, 1994.

Okamoto, Demetrick, Spillare, Hagiwara, Hussain, Bennett, Forrester, Gerwin, Serrano, Beach et al., “Mutations and altered expression of p16INK4 in human cancer,” Proc Natl Acad Sci USA, 1(23):11045-11049, 1994.

Omitz, Hammer, Davison, Brinster, and Palmiter, “Promoter and enhancer elements from the rat elastase i gene function independently of each other and of heterologous enhancers,” Mol. Cell. Biol. 7:3466, 1987.

Ondek, Sheppard, and Herr, “Discrete Elements Within the SV40 Enhancer Region Display Different Cell-Specific Enhancer Activities,” EMBO J., 6:1017, 1987.

O'Neil et al., “Effects of estrogen on leptin gene promoter activation in MCF-7 breast cancer and JEG-3 choriocarcinoma cells:selective regulation via estrogen receptors alpha and beta”. Mol. Bio. Endocrinol. 15:176(1-2):67-75.

Orlow, Lianes, Lacombe, Dalbagni, Reuter, Cordon-Cardo, “Chromosome 9 allelic losses and microsatellite alterations in human bladder tumors,”, Cancer Res, 54(11):2848-2851, 1994.

Palmiter, Chen, and Brinster, “Differential regulation of met allothionein-thymidine kinase fusion genes in transgenic mice and their offspring,” Cell, 29:701, 1982.

Paskind, Weinberg, Baltimore, “Dependence of Moloney murine leukemia virus production on cell growth,” Virology, 67:242-248, 1975.

Pech, Rao, Robbins, and Aaronson, “Functional identification of regulatory elements within the promoter region of platelet-derived growth factor 2,” Mol. Cell. Biol., 9:396, 1989.

Perez-Stable and Constantini, “Roles of fet al y-globin promoter elements and the adult β-globin 3′ enhancer in the stage-specific expression of globin genes,” Mol. Cell. Biol., 10:1116, 1990.

Picard and Schaffner, “A lymphocyte-specific enhancer in the mouse immunoglobulin kappa gene,” Nature, 307:83, 1984.

Pinkert, Omitz, Brinster, and Palmiter, “An albumin enhancer located 10 kb upstream functions along with its promoter to direct efficient, liver-specific expression in transgenic mice,” Genes and Dev., 1:268, 1987.

Ponta, Kennedy, Skroch, Hynes, and Groner, “Hormonal Response Region in the Mouse Mammary Tumor Virus Long Terminal Repeat Can Be Dissociated From the Proviral Promoter and Has Enhancer Properties,” Proc. Natl. Acad. Sci. U.S.A., 82:1020, 1985.

Porton, Zaller, Lieberson, and Eckhardt, “Immunoglobulin heavy-chain enhancer is required to maintain transfected .gamma.2a gene expression in a pre-b-cell line,” Mol. Cell. Biol., 10: 1076, 1990.

Potter, Weir, Leder, “Enhancer-dependent expression of human kappa immunoglobulin genes introduced into mouse pre-B lymphocytes by electroporation,” Proc. Nat'l Acad. Sci. USA, 81:7161-7165, 1984.

Queen and Baltimore, “Immunoglobulin gene transcription is activated by downstream sequence elements,” Cell, 35:741, 1983.

Quinn, Farina, Gardner, Krutzsch, and Levens, “Multiple components are required for sequence recognition of the ap1 site in the gibbon ape leukemia virus enhancer,” Mol. Cell. Biol., 9:4713, 1989.

Ravindranath and Morton, “Role of gangliosides in active immunotherapy with melanoma vaccine,” Intern. Rev. Immunol., 7: 303-329, 1991.

Redondo, Hata, Brocklehurst, and Krangel, “A T-cell-specific transcriptional enhancer within the human T-cell receptor .delta locus,” Science, 247:1225, 1990.

Reisman and Rotter, “Induced expression from the moloney murine leukemia virus long terminal repeat during differentiation of human myeloid cells is mediated through its transcriptional enhancer,” Mol. Cell. Biol., 9:3571, 1989.

Remington's Pharmaceutical Sciences, 15th Edition, Chapter 61, pages 1035-1038 and 1570-1580.

Resendez Jr., Wooden, and Lee, “Identification of highly conserved regulatory domains and protein-binding sites in the promoters of the rat and human genes encoding the stress-inducible 78-kilodalton glucose-regulated protein,” Mol. Cell. Biol., 8:4579, 1988.

Richard, Schumacher, Royer-Pokora, Roberts, “Par4 is a coactivator for a splice isoform-specific transcriptional activational domain in WT1, ” Genes Dev., 15:328-339, 2001.

Ridgeway, “Mammalian expression vectors,” In: Rodriguez R L, Denhardt D T, (ed.) Vectors: A survey of molecular cloning vectors and their uses. Stoneham: Butterworth, pp. 467-492, 1988.

Ripe, Lorenzen, Brenner, and Breindl, “Regulatory elements in the 5′ flanking region and the first intron contribute to transcriptional control of the mouse alpha-1-type collagen gene,” Mol. Cell. Biol., 9:2224, 1989.

Rippe, Brenner and Leffert, “DNA-mediated gene transfer into adult rat hepatocytes in primary culture,” Mol. Cell Biol., 10:689-695, 1990.

Rittling, Coutinho, Amarm, and Kolbe, “AP-1/jun-binding sites mediate serum inducibility of the human vimentin promoter,” Nuc. Acids Res., 17:1619, 1989.

Rosen, Sodroski, and Haseltine, “The location of cis-acting regulatory sequences in the human t-cell lymphotropic virus type III (HTLV-111/LAV) long terminal repeat,” Cell, 41:813, 1988.

Rosenberg et al., N. Engl. J. Med., 319:1676, 1988.

Sakai, Helms, Carlstedt-Duke, Gustafsson, Rottman, and Yamamoto, “Hormone-Mediated Repression: A Negative Glucocorticoid-Response Element From the Bovine Prolactin Gene,” Genes and Dev., 2:1144, 1988.

Sambrook et. al., In: Molecular Cloning. A Laboratory Manual, 2d Ed., Cold Spring Harbor Laboratory Press, Cold Spring Harbor, N.Y., 1989.

Satake, Furukawa, and Ito, “Biological activities of oligonucleotides spanning the f9 point mutation within the enhancer region of polyoma virus DNA,” J. Virology, 62:970, 1988.

Schaffner, Schirm, Muller-Baden, Wever, and Schaffner, “Redundancy of Information in Enhancers as a Principle of Mammalian Transcription Control,” J. Mol. Biol., 201:81, 1988.

Searle, Stuart, and Palmiter, “Building a metal-responsive promoter with synthetic regulatory elements,” Mol. Cell. Biol., 5:1480, 1985.

Serrano, Gomez-Lahoz, DePinho, Beach, Bar-Sagi, “Inhibition of ras-induced proliferation and cellular transformation by p16INK4,” Science, 267(5195):249-252, 1995.

Serrano, Hannon, Beach, “A new regulatory motif in cell-cycle control causing specific inhibition of cyclin D/CDK4,” Nature, 366:704-707, 1993.

Sharp and Marciniak, “HIV Tar: an RNA Enhancer?,” Cell, 59:229, 1989.

Shaul and Ben-Levy, “Multiple Nuclear Proteins in Liver Cells are Bound to Hepatitis B Virus Enhancer Element and its Upstream Sequences,” EMBO J., 6:1913, 1987.

Sherman, Basta, Moore, Brown, and Ting, “Class II Box Consensus Sequences in the HLA-DR.alpha. Gene: Transcriptional Function and Interaction with Nuclear Proteins,” Mol. Cell. Biol.,9:50, 1989.

Silberstein, Van Horn, Strickland, Roberts, Daniel, “Altered expression of the WT1 wilms tumor suppressor gene in human breast cancer,” Proc. Natl. Acad. Sci. USA, 94:8132-8137, 1997.

Sleigh and Lockett, “SV40 Enhancer Activation During Retinoic-Acid-Induced Differentiation of F9 Embryonal Carcinoma Cells,” J. EMBO, 4:3831, 1985.

Spalholz, Yang, and Howley, “Transactivation of a Bovine Papilloma Virus Transcriptional Regulatory Element by the E2 Gene Product,” Cell, 42:183, 1985.

Spandau and Lee, “Trans-Activation of Viral Enhancers by the Hepatitis B Virus X Protein,” J. Virology, 62:427, 1988.

Spandidos and Wilkie, “Host-Specificities of Papilloma Virus, Moloney Murine Sarcoma Virus and Simian Virus 40 Enhancer Sequences,” EMBO J., 2:1193, 1983.

Stephens and Hentschel, “The Bovine Papilloma Virus Genome and its Uses as a Eukaryotic Vector,” Biochem. J., 248:1, 1987.

Stratford-Perricaudet and Perricaudet, In: Human Gene Transfer, Eds, O. Cohen-Haguenauer and M. Boiron, Editions John Libbey Eurotext, France, pp. 51-61, 1991.

Stuart, Searle, and Palmiter, “Identification of Multiple Met al Regulatory Elements in Mouse Met allothionein-I Promoter by Assaying Synthetic Sequences,” Nature, 317:828, 1985.

Sukhatme, Cao, Chang, Tsai-Morris, Stamenkovich, Ferreira, Cohen, Edwards, Shows, Curran et al., “A zinc finger-encoding gene coregulated with c-fos during growth and differentiation, and after cellular depolarization,” Cell, 53:37-43, 1988.

Sullivan and Peterlin, “Transcriptional enhancers in the HLA-DQ subregion,” Mol. Cell. Biol., 7:3315, 1987.

Sutherland, Hall, Pang, Musgrove, Clarke, “Effect of medroxyprogesterone acetate on proliferation and cell cycle kinetics of human mammary carcinoma cells,” Cancer Res., 48:5084-5091, 1988.

Swartzendruber and Lehman, “Neoplastic Differentiation: Interaction of Simian Virus 40 and Polyoma Virus with Murine Teratocarcinoma Cells,” J. Cell. Physiology, 85:179, 1975.

Szoka and Papahadjopoulos, “Procedure for preparation of liposomes with large internal aqueous space”, Proc. Natl. Acad. Sci., 1978, 75:4194-4198.

Takebe, Seiki, Fujisawa, Hoy, Yokota, Arai, Yoshida, and Arai, “SRα promoter: an efficient and versatile mammalian cDNA expression system composed of the simian virus 40 early promoter and the R-U5 segment of human T-cell leukemia virus type 1 long terminal repeat,” Mol. Cell. Biol., 8:466, 1988.

Tavernier, Gheysen, Duerinck, Can Der Heyden, and Fiers, “Deletion mapping of the inducible promoter of human IFN-beta gene,” Nature, 301:634, 1983.

Taylor and Kingston, “E1A Trans-Activation of Human HSP70 Gene Promoter Substitution Mutants is Independent of the Composition of Upstream and TATA Elements,” Mol. Cell. Biol., 10:176, 1990b.

Taylor and Kingston, “Factor Substitution in a Human HSP70 Gene Promoter: TATA-Dependent and TATA-Independent Interactions,” Mol. Cell. Biol., 10:165, 1990a.

Taylor, Solomon, Weiner, Paucha, Bradley, and Kingston, “Stimulation of the Human Heat-Shock Protein 70 Promoter in vitro by Simian Virus 40 Large T Antigen,” J. Biol. Chem., 264:15160, 1989.

Temin, “Retrovirus vectors for gene transfer: Efficient integration into and expression of exogenous DNA in vertebrate cell genome,” In: Gene Transfer, Kucherlapati (ed.), New York: Plenum Press, pp. 149-188, 1986.

Thiesen, Bosze, Henry, and Charnay, “A DNA Element Responsible for the Different Tissue Specificities of Friend and Moloney Retroviral Enhancers,” J. Virology, 62:614, 1988.

Toyooka, Tanaka, Hirota, Tanaka, Takagi, Yamanouchi, tojo, Tachi, “Wilms' tumor suppressor gene (WT1) as a target gene of SRY function in a mouse ES cell line transfected with SRY,” Int. J Dev. Biol., 42:1143-1151, 1998.

Treisman, “Transient Accumulation of c-fos RNA Following Serum Stimulation Requires a Conserved 5′ Element and c-fos 3′ Sequences,” Cell, 42:889, 1985.

Tronche, Rollier, Bach, Weiss, and Yaniv, “The Rat Albumin Promoter: Cooperation with Upstream Elements is Required When Binding of APF/HNF 1 to the Proximal Element is Partially Impaired by Mutation or Bacterial Methylation,” Mol. Cell. Biol., 9:4759, 1989.

Tronche, Rollier, Herbomel, Bach, Cereghini, Weiss, and Yaniv, “Anatomy of the Rat Albumin Promoter,” Mol. Biol. Med., 7:173, 1990.

Trudel and Constantini, “A 3′ Enhancer Contributes to the Stage-Specific Expression of the Human Beta-Globin Gene,” Genes and Dev., 6:954, 1987.

Tsujimoto and Croce, “Analysis of the structure, transcripts, and protein products of bc1-2, the gene involved in human follicular lymphoma,” Proc Natl Acad Sci U S A. 83(14):5214-5218, 1986.

Tsujimoto, Cossman, Jaffe, Croce, “Involvement of the bc1-2 gene in human follicular lymphoma,” Science, 228(4706): 1440-1443, 1985.

Tur-Kaspa, Teicher, Levine, Skoultchi and Shafritz, “Use of electroporation to introduce biologically active foreign genes into primary rat hepatocytes,” Mol Cell Biol., 6:716-718, 1986.

Tyndall, La Mantia, Thacker, Favaloro, and Kamen, “A Region of the Polyoma Virus Genome Between the Replication Origin and Late Protein-Coding Sequences is Required in cis for Both Early Gene Expression and Viral DNA Replication,” Nuc. Acids. Res., 9:6231, 1981.

Vannice and Levinson, “Properties of the Human Hepatitis B Virus Enhancer: Position Effects and Cell-Type Nonspecificity,” J. Virology, 62:1305, 1988.

Vasseur, Kress, Montreau, and Blangy, “Isolation and Characterization of Polyoma Virus Mutants Able to Develop in Multipotential Murine Embryonal Carcinoma Cells,” Proc Natl. Acad. Sci. U.S.A., 77:1068, 1980.

Wagner et al., Science, 260:1510-1513, 1993.

Walker et al., “Butyrate, a histone deacetylase inhibitor, activates the human IGF binding protein-3 promoter in breast cancer cells: molecular mechanism involves an SP1/SP3 multiprotein complex.” Endocrinology, 142(9):3187-27.

Wang and Calame, “SV40 enhancer-binding factors are required at the establishment but not the maintenance step of enhancer-dependent transcriptional activation,” Cell, 47:241, 1986.

Wang, Qiu, Huang, Gurrieri, Deuel, “Products of alternatively spliced transcripts of the Wilms' tumor suppressor gene, wt1, have altered DNA binding specificity and regulate transcription in different ways,” Oncogene, 10:415-422, 1995.

Wang, Santini, Qin, Huang, “A Mg²⁺-dependent, Ca²⁺-inhibitable serine/threonine protein phosphatase from bovine brain,” J. Biol. Chem., 270:25607-25612, 1995.

Weber, De Villiers, and Schaffner, “An SV40 ‘Enhancer Trap’ Incorporates Exogenous Enhancers or Generates Enhancers From its Own Sequences,” Cell, 36:983, 1984.

Weinberg, “Tumor suppressor genes,” Science, 254(5035):1138-1146, 1991.

Weinberger, Jat, and Sharp, “Localization of a Repressive Sequence Contributing to B-cell Specificity in the Immunoglobulin Heavy-Chain Enhancer,” Mol. Cell. Biol., 8:988, 1984.

Winoto and Baltimore, “α:β-lineage-specific Expression of the α T-Cell Receptor Gene by Nearby Silencers,” Cell, 59:649, 1989.

Wong, Nicolau, Hofschneider, “Appearance of β-lactamase activity in animal cells upon liposome mediated gene transfer,” Gene, 10:87-94, 1980.

Wu and Wu, “Receptor-mediated in vitro gene transfections by a soluble DNA carrier system,” J. Biol. Chem., 262:4429-4432, 1987.

Wu and Wu, “Evidence for targeted gene delivery to HepG2 hepatoma cells in vitro,” Biochemistry, 27:887-892, 1988.

Yamagami, Sugiyama, Inoue, Ogawa, Tatakawa, Hirata, Kudoh, Akiyama, Murakami, Maekawa, “Growth inhibition of human leukemic cells by WI1 (Wilms tumor gene) antisense oligodeoxynucleotides: Implications for the involvement of WT1 in leukeomogenesis,” Blood, 87:2978-2884, 1996.

Yang, Burkholder, Roberts, Martinell and McCabe, “In vivo and in vitro gene transfer to mammalian somatic cells by particle bombardment,” Proc Nat'l Acad Sci. USA, 87:9568-9572, 1990.

Yutzey, Kline, and Konieczny, “An Internal Regulatory Element Controls Troponin I Gene Expression,” Mol. Cell. Biol., 9:1397, 1989.

Zelenin et al., “High-velocity mechanical DNA transfer of the chloramphenicol acetyltransferase gene into rodent liver, kidney and mammary gland cells in organ explants and in vivo,” FEBS Lett., 280:94-96, 1991.

What is claimed is:

1. A method of inhibiting the growth of a breast cancer cell expressing a Wilms' Tumor 1 (WT1) gene product comprising contacting said cell with an amount of a WT1 antisense molecule effective to inhibit the growth of the breast cancer cell.

2. The method of claim 1, wherein said WT1 antisense molecule is a DNA.

3. The method of claim 1, wherein said WT1 antisense molecule is an RNA.

4. The method of claim 1 wherein the antisense molecule is produced from an expression vector encoding said antisense under the control of a promoter active in said cell.

5. The method of claim 4, wherein said promoter is a constitutive promoter.

6. The method of claim 5, wherein said constitutive promoter is a CMV promoter, an RSV promoter, an SV40 promoter.

7. The method of claim 4, wherein said promoter is a tissue specific promoter.

8. The method of claim 7, wherein said tissue specific promoter is leptin gene promoter, IGF binding protein-3 promoter, adenomatous polyposis coli gene promoter.

9. The method of claim 4, wherein said promoter is an inducible promoter.

10. The method of claim 9, wherein said inducible promoter is Tet-On system, Tet-Off system.

11. The method of claim 1, wherein said breast cancer cell is estrogen receptor-positive.

12. The method of claim 1, wherein said breast cancer cell is estrogen receptor-negative.

13. The method of claim 2, wherein said DNA is an oligonucleotide.

14. The method of claim 13, wherein said oligonucleotide is 6 to about 50 bases in length.

15. The method of claim 13, wherein said oligonucleotide comprises one or more modifed bases.

16. The method of claim 1, wherein said antisense molecule hybridizes to a WT1 transcript.

17. The method of claim 16, wherein said antisense molecule hybridizes to a translation initiation site or a splice site.

18. The method of claim 1, wherein said antisense molecule hybridizes to a WT1 genomic sequence.

19. The method of claim 18, wherein said antisense molecule hybridizes to a transcription start site, an intron, an exon, or an intron-exon junction.

20. The method of claim 2, wherein said DNA is a double-stranded DNA.

21. The method of claim 2, wherein said DNA is a single-stranded DNA.

22. The method of claim 4, wherein said expression vector is a non-viral vector.

23. The method of claim 4, wherein said expression vector is a viral vector.

24. The method of claim 23, wherein said viral vector is selected from the group consisting of adenovirus, retrovirus, herpesvirus, vaccinia virus, adeno-associated virus, lentivirus and polyoma virus.

25. The method of claim 1, wherein said antisense molecule is associated with one or more lipid.

26. The method of claim 25, wherein said antisense molecule is encapsulated in a liposome.

27. The method of claim 25, wherein the lipid comprises at least one neutrally charged lipid.

28. The method of claim 27, wherein said neutrally charged lipid is DOPC.

29. The method of claim 25, further defined as comprising more than one lipids wherein the lipids on a whole are neutrally charged.

30. The method of claim 17, wherein said antisense molecule hybridizes to a translation initiation site and comprises 5′-GTCGGAGCCCATTTGCTG-3′.

31. The method of claim 30, wherein said antisense molecule consists of 5′-GTCGGAGCCCATTTGCTG-3′.

32. The method of claim 1, wherein said cell expresses multiple WT1 isoforms.

33. The method of claim 1, wherein said cell expresses one or more adverse oncogene products.

34. A method of treating a subject having a breast cancer tumor, cells of which express a Wilms' Tumor 1 (WT1) gene product, comprising administering to said subject an effective amount of a WT1 antisense molecule.

35. The method of claim 34, wherein said antisense molecule is administered to said tumor by intratumoral injection.

36. The method of claim 34, wherein said antisense moleclue is administered to the tumor vasculature.

37. The method of claim 34, wherein said antisense molecule is administered locally to said tumor.

38. The method of claim 34, wherein said antisense molecule is administered regionally to said tumor.

39. The method of claim 34, wherein said antisense molecule is administered to the lymphatic system locally or regionally to said tumor.

40. The method of claim 34, further comprising administering to said subject a second breast cancer therapy.

41. The method of claim 40, wherein said second breast cancer therapy is chemotherapy, radiation therapy, immunotherapy, hormonal therapy, or gene therapy.

42. The method of claim 40, wherein said second breast cancer therapy is provided to said subject prior to said WT1 antisense molecule.

43. The method of claim 40, wherein said second breast cancer therapy is provided to said subject after said WT1 antisense molecule.

44. The method of claim 40, wherein said second breast cancer therapy is provided to said subject at the same time as said WT1 antisense molecule.

45. A method of predicting breast cancer progression in a subject having breast cancer comprising:
(a) obtaining a sample from said subject comprising breast cancer tumor cells; and
(b) assessing expression of one or more isoforms of Wilms' Tumor 1 (WT1) gene product in said cells.

46. The method of claim 45, wherein assessing comprises measuring WT1 protein levels.

47. The method of claim 44, wherein measuring comprises quantitative immunodetection.

48. The method of claim 45, wherein assessing comprises measuring WT1 mRNA levels.

49. The method of claim 48, wherein measuring comprises quantitative PCR.

50. A method of screening a candidate substance for activity against breast cancer comprising:
(i) providing a cell that expresses one or more isoforms of the Wilms' Tumor 1 (WT1) gene product;
(ii) contacting the cell with the candidate substance suspected of inhibiting WT1; and
(iii) measuring the effect of the candidate substance on the cell.
wherein a decrease in the amount of WT1 gene product in said cell, as compared to a cell not treated with said candidate substance, indicates that said candidate substance has activity against breast cancer.

51. The method of claim 50, wherein said candidate substance is a protein, a nucleic acid or a small molecule pharmaceutical.

52. The method of claim 50, wherein measuring comprises determining the level of a WT1 gene product in said cell.

53. The method of claim 50, wherein said cell is a breast cancer cell.


SEQUENCE LISTING (2 sequences)

SEQ ID NO: 1 - DNA, 18 bp, Artificial Sequence
  Description of Artificial Sequence  Synthetic Primer
gtcggagcccatttgctg

SEQ ID NO: 2 - DNA, 18 bp, Artificial Sequence
  Description of Artificial Sequence  Synthetic Primer
gggcttttgaactctgct